SEQUENCE LISTING (149 sequences)

SEQ ID NO: 1 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 6 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 7 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 8 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 9 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 10 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 11 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 12 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 13 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 14 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 15 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 16 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 17 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 18 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 19 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 20 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 21 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 22 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 23 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 24 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 25 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 26 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 27 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 28 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 29 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 30 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 31 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 32 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 33 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 34 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 35 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 36 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 37 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 38 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 39 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 40 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 41 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 42 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 43 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 44 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 45 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 46 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 47 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 48 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 49 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 50 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 51 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 52 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 53 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 54 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 55 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 56 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 57 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 58 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 59 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 60 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 61 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 62 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 63 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 64 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 65 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 66 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 67 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 68 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 69 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 70 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 71 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 72 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 73 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 74 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 75 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 76 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 77 - DNA, 667 bp, Arabidopsis thaliana
  misc_feature (1)..(857): Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 78 - DNA, 1686 bp, Arabidopsis thaliana
  misc_feature (1)..(1686): Ceres CLONE ID no. 110454
aaaaaggatcaaacatagtatagcttggactctttttgccgtctatgatctctctcaatctctcatcttagctttctttctctctctctttgtgtcttcatttgtttatctcatcaatggattcaaattttcattactcgatagatcttaacgaagatcaaaaccatcacgaacaaccctttttctatcctcttggatcctcttcctcgcttcatcatcatcatcatcatcatcatcatcaagtcccttctaattcttcatcttcttcttcgtccatttcatcgctctcctcttacctccctttcttgatcaactctcaagaagatcaacatgttgcctacaacaacacttatcacgctgatcatctccatctttctcaacccctcaaggccaagatgtttgtggctaacggtggatcatcatcatgcgatcacatggtgccaaagaaggagacaagactgaaactaacgataaggaaaaaagatcacgaagaccaaccccatcctcttcatcaaaacccgacaaaacccgattcagactccgacaagtggttgatgtccccaaagatgcggttgatcaagaaaacaatcaccaacaataaacagctcactgatcagactaataataataatcataaagaaagtgatcactaccctttgaatcataagactaatttcgacgaggatcaccatgaagatcttaatttcaagaacgtcttgaccaggaagaccacggccgcgaccaccgagaatcgctacaatacaatcaacgagaacggttatagtaataacaatggcgtgattagggtttgttcggattgtaacaccaccaagactcctctttggcgaagtggacctcgaggtcccaagtctctttgtaacgcatgtggtatacggcaaagaaaggcaaggcgagccgctatggccgcggccgctgcagccggcgaccaagaggtggcggtagcgccccgagtgcaacaattaccgctgaaaaagaagttgcaaaataaaaaaaaagagatcaaacggaggggaaaaatacaatcactctcctccaatggtggccaaggccaaaaagtgcaagatcaaagaggaagaggagaaggaaatggaagcggaaacggttgccggagattcagagatcagcaaatctacaacttcttctaattcttcgatttcgtcaaacaaattttgcttcgatgatttgacaataatgttgagcaaaagctcagcttatcaacaagtgttcccacaagatgagaaggaggctgctgttttgctcatggctctgtcgtatggaatggttcacggttgatcagatcatcacaatatcctcattacaaaaaggtttattttaatagtaatatatagattatagtaatcataataatgattgattgttaaatcttggagtgattagtttagtttttgcagttggtctaatatcaggagtcaaaacattttataataagagtgtgtgagagtttaatatgataattaatagctctatatatgtttatggagattttggttttgagtttgttgtgtttttgttttccgatggaacgaacattagtcacccgataatgtgggtaattcttgttcatttacaagttttatagtactgtattgtgtaacgaggttggagaaataatataatcaattaagggtaaaaaaaaaaaaaaaa

SEQ ID NO: 79 - protein, 349 aa, Arabidopsis thaliana
  misc_feature (1)..(349): Ceres CLONE ID no. 110454
MDSNFHYSIDLNEDQNHHEQPFFYPLGSSSSLHHHHHHHHHQVPSNSSSSSSSISSLSSYLPFLINSQEDQHVAYNNTYHADHLHLSQPLKAKMFVANGGSSSCDHMVPKKETRLKLTIRKKDHEDQPHPLHQNPTKPDSDSDKWLMSPKMRLIKKTITNNKQLTDQTNNNNHKESDHYPLNHKTNFDEDHHEDLNFKNVLTRKTTAATTENRYNTINENGYSNNNGVIRVCSDCNTTKTPLWRSGPRGPKSLCNACGIRQRKARRAAMAAAAAAGDQEVAVAPRVQQLPLKKKLQNKKKEIKRRGKIQSLSSNGGQGQKVQDQRGRGEGNGSGNGCRRFRDQQIYNFF

SEQ ID NO: 80 - protein, 347 aa, Oryza sativa subsp. japonica
  misc_feature (1)..(347): Public GI no. 54291240
MSTIYMSQLSAALPLMEGEHHHHHQDHHQGHFQAFSLQPKDPPVLFPFVISRRSSSSSPSDSTTLSYGSDHHLTQQQQHQHQAMLEPQNMIGGSSAGIFATPFPTVKSIRDDMIERSQFDPYDTEKLQASCGLAKVVAGGKWSAVPAAKMKITRKMGEPSSGVTGGAATTVAPKKPRRRPAQAYEDHGHGGAMGQAFGVIRVCSDCNTTKTPLWRSGPCGPKSLCNACGIRQRKARRAMMASGLPASPNAAGPKAAAHSGATNAAAAAAMEETAESATVAPPPAPTTRGGTLVDSIGLSWSKTHAAATASCSFRPSPVAPGFAAAVQDEITDAAMLLMTLSCGLVRS

SEQ ID NO: 81 - DNA, 771 bp, Arabidopsis thaliana
  exon (1)..(66)
  Intron (67)..(432)
  exon (433)..(771)
  misc_feature (1)..(771): Ceres LOCUS ID no. At1g16640
atggctgacactggtgaggttcaatttatgaagcctttcatctcagaaaagtcatctaaatcattggtgagcatggctctctatctttcttcttttttattttcttatttcaacaagttcaaaatatgtgtttagtgttctgatttggagacttatggtttagggtaaattgctttagttcattttcatggagctaataacttaggtcctttaattgcttttgttagctttaaattagcaaaaagcaagtgaaagcttactgaatcttgctagatttagaatgaagtttatgtgttggtgttgttataggctgcttgaaccaaacatttctgcggggctgaaaagtgaaaaatagcaagtccattgtttctacgttgctgctcttggaatgtctcctgagagttgtttttttatatataaatgtttgattaggaaatccccttgggattcaacgaatacttcccagcaccatttccaataactgtggacctcctggactatagtgggagatcttggacggttaggatgaagaaaagaggagagaaagtgtttcttacagttggttgggagaattttgtaaaagataacaatctcgaagatggcaagtatttgcagttcatctatgaccgcgacaggaccttttatgttatcatatatggtcataacatgtgtagcgaatatagagacttccctcaagtcgcagtagaggttgatgactatgaaaacggtgaagaagaagaagacggcgacgatcaagacaaacatcagtaa

SEQ ID NO: 82 - DNA, 405 bp, Arabidopsis thaliana
  misc_feature (1)..(405): Ceres LOCUS ID no. At1g16640
atggctgacactggtgaggttcaatttatgaagcctttcatctcagaaaagtcatctaaatcattggaaatccccttgggattcaacgaatacttcccagcaccatttccaataactgtggacctcctggactatagtgggagatcttggacggttaggatgaagaaaagaggagagaaagtgtttcttacagttggttgggagaattttgtaaaagataacaatctcgaagatggcaagtatttgcagttcatctatgaccgcgacaggaccttttatgttatcatatatggtcataacatgtgtagcgaatatagagacttccctcaagtcgcagtagaggttgatgactatgaaaacggtgaagaagaagaagacggcgacgatcaagacaaacatcagtaa

SEQ ID NO: 83 - protein, 134 aa, Arabidopsis thaliana
  misc_feature (1)..(134): Ceres LOCUS ID no. At1g16640
MADTGEVQFMKPFISEKSSKSLEIPLGFNEYFPAPFPITVDLLDYSGRSWTVRMKKRGEKVFLTVGWENFVKDNNLEDGKYLQFIYDRDRTFYVIIYGHNMCSEYRDFPQVAVEVDDYENGEEEEDGDDQDKHQ

SEQ ID NO: 84 - protein, 134 aa, Brassica napus
  misc_feature (1)..(134): Ceres CLONE ID no. 1090611
MAAPDEDQRFIKPFISHKSAKSLAIPLAFNEYXPDPLPNTVELLDYYGRSWTIRMKKRGETVFLTVGWENFVKDNELEDGKMMEFIYDCDRTFYVVIFGHGGVSELRVFPQAVVDVGDYXTGEEEGEEEEEEEK

SEQ ID NO: 85 - DNA, 603 bp, Arabidopsis thaliana
  misc_feature (1)..(603): Ceres LOCUS ID no. At1g32690
atggacgaagatggtttttcacaatgttcaaaacacttttcaaaacgcggttattttagcggtgggggcgtttgtccattttgtctccacgaacgtctctcttctctttgtccagattgtgctcacgatcttccttgttcgtgtagctcacgcgccgccgtatctttctctccttcgtcttcttcgtcttcctcgtcgttctctatcttcgccggagatatcagttttaatatatccggcgttggatcggtgggccgagtcgcgagtttgattgaatgtgaaccggcgtttcggagatcgaaatctatggcgatcccgattaaaccggattcggttattgattccggtttggatcatggtaggagtaagaaaacgtcgtcgttttggagaatttttatgggaaacagaggagatactaaaccggctattatgaagaaatcaagatccgttgcggtcgccggagaatttggattttctccggttccggttccggcgaccgggaagggtaaaggctggaattttccgagtccgatcaaggttttccggcaatctcgagtctcgaagatgatatttcaacaacggtctccattgtatagaggttga

SEQ ID NO: 86 - protein, 200 aa, Arabidopsis thaliana
  misc_feature (1)..(200): Ceres LOCUS ID no. At1g32690
MDEDGFSQCSKHFSKRGYFSGGGVCPFCLHERLSSLCPDCAHDLPCSCSSRAAVSFSPSSSSSSSSFSIFAGDISFNISGVGSVGRVASLIECEPAFRRSKSMAIPIKPDSVIDSGLDHGRSKKTSSFWRIFMGNRGDTKPAIMKKSRSVAVAGEFGFSPVPVPATGKGKGWNFPSPIKVFRQSRVSKMIFQQRSPLYRG

SEQ ID NO: 87 - protein, 235 aa, Glycine max
  misc_feature (1)..(235): Ceres CLONE ID no. 706980
MALYMDEEEIWKCPKHPSKRRRSGICPTCLRDRLVTLCPDCANVRPCSCYATSSSSSSSSSSSLSRFSGAGDGVGSVGRVHNLIEQEPGLRRSRSMAIPFLRSRSRFSGGGDRVSDLDSTRDSPAMNGSRSARSFWSMFKSQKSSRHGGGGGGAPEQEWEAKKILAEERDGDGGINPMMVRSRSVAVTAVTVVSGDGELRPRTKGRGWFFPSPMKAFRQSKVSKVIQEGSPLYRG

SEQ ID NO: 88 - DNA, 831 bp, Populus balsamifera subsp. trichocarpa
  misc_feature (1)..(831): Ceres ANNOT ID no. 1485507
atggttaacatgtgtagtcatattataaaacctgagagccaaatagagaagtcagaacaggatagcatttttccagagagagagagaggaaaaggcaaaaaggaaagaagaaattctttgttagagggagggggatctacacaggaaacttacaacaaaatggctctttatgttgatgaagaagagatatggaaatgtttaaagcacccttccaagcgtcgccgaacaggaatctgccacgtgtgcctccgcgaacgtctctcctctctttgccctgactgcgctagcgcgcgcccctgcacttgctacgccaccaccgcgtcctcctcttctggcaccacttcatcctcctcctcccactgcttctcctccgcttccggaatcggaagcgtcggtcgagtctccaatttaatcgagagcgaacccgcttttcgtcgctctcgatccctagctgttccgtttctccggtcaaagccatcggctgaccatagttataacaaccacaaagcgtcctcgtcgttctggtcattgttcaagggagggcatggtaacaggagcatgagagaggaggtcgaaagacgacacgtggtgattttgaaggaggaggagctggaggaatcatcgaggaaggtaaatgaagatgaggagaggaggaggatgatgaggaagtcaagatcggtggcggtgacttcagagtcaagaggaagtgacgtgaggagatcgtcaaagggagggaagggatggtatttccctagtccgatcaaggttttcaagcaatcgatctcgagagggattttggcgcatgaaaggtcgcctttgtatagaggttga

SEQ ID NO: 89 - protein, 223 aa, Populus balsamifera subsp. trichocarpa
  misc_feature (1)..(223): Ceres ANNOT ID no. 1485507
MALYVDEEEIWKCLKHPSKRRRTGICHVCLRERLSSLCPDCASARPCTCYATTASSSSGTTSSSSSHCFSSASGIGSVGRVSNLIESEPAFRRSRSLAVPFLRSKPSADHSYNNHKASSSFWSLFKGGHGNRSMREEVERRHVVILKEEELEESSRKVNEDEERRRMMRKSRSVAVTSESRGSDVRRSSKGGKGWYFPSPIKVFKQSISRGILAHERSPLYRG

SEQ ID NO: 90 - DNA, 1097 bp, Panicum virgatum
  misc_feature (156)..(761): Encodes the peptide sequence given in SEQ ID NO. 91
cttttccaacagcgacgcaacgagctcgtaacgcccgtaccctcgcacgcgcgcagagagagagagagagagagagagagagagagcaagtgagcagagaagaagaggaaggagcgagaagcctgggaagggagagcgagcgcaaccgaatcgatatgaagccggagaaggcagcggtggccgtggcagccggcggcggcggcgacgagtggcggtgccggaagcacccggccgcgccgagatccggcggtggggtgtgcccgtactgcctccgcgaccggctcctccgcctgtgccccaactgcgcacgcgtgcggccctgcccctgcgccgcgtcgtgcgcctccccgtcctcctcctcggcgtccggcgacgcggtgggccgcgtgcacagcctcatcgagcgggagcaccggatcgcgcgctcgcggtccgtggccgcgggctcctccgccgcgttcgtcgccgccgtgggtgccccctccgcggcggggcccacctccgggggcggcaggcggaaggcgcgcgtctgggggtggccgccgttctggaagcccgcgtcgcgggacggggacgcggggatgggggaggacgaggaggaggggctgggcctgccgcgctcgagctccgtgtccgcgacggccgtggagaccaagaccgcggcggcggcggcgcgcgcgaggtgggggtggcacttcccgagcccgctgaaggcgttccggcaccggaggtcgtcggcgagcgtggccgggcgggcgtgagcgccaaggtacaccgcccccttctggagatggccggaaaagcgattcgcgggtgggggtcatgtggccggggatggttaattattaattttaactgtataaggcggtggattagaggaggagcgttggatctcctttttctccttttgttgtggcgtgccgctaattccatgattaagttagacggggatgcgaaagcctgcgatctgggcgggatgtgtgcatatgctagtgggtgatctcctcatggcttcccttcaccttttgtagtgtgcttgtacccgactctgtaacacaccaaattggatattttaagctctccaatttgttctggct

SEQ ID NO: 91 - protein, 201 aa, Panicum virgatum
  misc_feature (1)..(201): Ceres CLONE ID no. 1822715
MKPEKAAVAVAAGGGGDEWRCRKHPAAPRSGGGVCPYCLRDRLLRLCPNCARVRPCPCAASCASPSSSSASGDAVGRVHSLIEREHRIARSRSVAAGSSAAFVAAVGAPSAAGPTSGGGRRKARVWGWPPFWKPASRDGDAGMGEDEEEGLGLPRSSSVSATAVETKTAAAAARARWGWHFPSPLKAFRHRRSSASVAGRA

SEQ ID NO: 92 - protein, 198 aa, Oryza sativa subsp. japonica
  misc_feature (1)..(198): Public GI no. 50911687
MKAEKAAVAVGGGGGGDEWRCRKHPVARSGGGVCPHCLRDRLLRLCPNCAHVRPCPCTCASPSSSSSASGEAVGRVHTLIEREHRIARSRSVAASSSLAAASTASATAGAVGGRRKARVWGWPPFWKPAAKDGVAAAAEEDEEEEGGMGLARSSSVSATVVEAKAAAAAAKARWGWHFPSPLKAFRHRRSSASMPERG

SEQ ID NO: 93 - DNA, 1015 bp, Arabidopsis thaliana
  misc_feature (1)..(1015): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcaaaaatagataaactcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 94 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 95 - DNA, 999 bp, Arabidopsis thaliana
  misc_feature (1)..(999): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaatcttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 96 - DNA, 1091 bp, Arabidopsis thaliana
  misc_feature (1)..(1091): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataagcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaaaaaaccaacttatatgagtaatagaaacgatcctcatattaggaattttagagattttctctcatctgtttcttaacttttcaatatttttatttttttaaattgtatgagtttctactaagaaactactgctggagttggtcttagcttcccaatgcttctccacctatatatatgcatatctccttcttaaaactccattgcactgggatccaacaatgtcctccgactcgtccaagatcaagaggaagcggaaccgcatcccttatactaaaggcgg

SEQ ID NO: 97 - DNA, 982 bp, Arabidopsis thaliana
  misc_feature (1)..(982): Ceres Promoter YP0080
aagcggcaatttagtaagaagtattcaatgtatcatttaccaaaagtatatggttttgggaagagttgttagggatgtattctttctaaacagatgatatgacgatgttcttgaaaactaatgttaaagacgggatctctcgcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaaaagaaaacaggaaatcaaatcaaaagataaagagaaggtaaaacaaaaggcaagaagcactaatgtttaatatttatagttttctccattaaagaaaaagcgatgatgtgtgtgttctcatcttttgtgaaagtatatatattgcttttgctcttctcaaaagcaaaagactcatccaacaacaacaacaaaaaaaaacctaaagctcaatccaaaagacgaagaatgcattggatactacaacttctttttcacttttctttcgaatttacaattatgattttcacaatacagtgtattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgttttt

SEQ ID NO: 98 - DNA, 1000 bp, Arabidopsis thaliana
  misc_feature (1)..(1000): Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 99 - DNA, 389 bp, Arabidopsis thaliana
  misc_feature (1)..(389): Ceres Promoter PD0901
cgcaggcccgatcggcccaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatatatatacacgtaataagaaggtgaatgaatctcacagcttacttgttctaaggcttccaataacgaaaacagtaggccggcaaggccaatc

SEQ ID NO: 100 - DNA, 3000 bp, Arabidopsis thaliana
  misc_feature (1)..(3000): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtcaatgtcaagcatacagctaaaatatcattatctaatattaagagtaaaacaagataattaaaaattgaaacaacaccatatttttatagctttacttatcgtatttttctagtcttcatggtaattgtgttgctttattttgtttataaatgaatttggttcgaccagatagtctaatatcagtttttaaacactggttttaataaaatcatatgtcggcaattcaacctgttacgttgtatgattgtatcctagtcaaataggggaggaggtactagtcgtttcaattagtttacgtaatcaatccaaagaaactataagctataaagatcctcaatttgttggttacaataaaaacaacagttgtcaaaatttatgtttataaaaagtaataactatgttccttcccatatagagcaaagtacctcaggataggcaaaccgtacttaatagcccttattcataatttgatccaactcttccccacaaaattgcaactgatgaagtcaatacttgtatagtgagtcaagctataaatgtctagtgatagttttgtctcttaaaaggttaacaaaagttatgacaagctgaaaaatcagagtttgctaggagtattacttacagttatcagtttaagtatcacatttatagtattgtatacaatgattcttaaattccaccttttccgtgcgaaaccaaattttctattggaaacatagaatgtaaacaaaaatatgggacgttgtccgttccaacattaaccaaacttgtctattactaatattcgtgttggtttgatgttggatgtctaaattcgttgaatcatgtgtctcttgacgaaatatgcatcttcttatttcttagtatagatgcactttatcattcttttagtacatgcttaatttttttttttaaaatatgttgattgtcatattgccaaaagtatgaattaaagacgcacatctaacacaagttagcagccgtaaatccttccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 101 - protein, 398 aa, Arabidopsis thaliana
  misc_feature: Public GI ID no.15241967
MDSNFHYSIDLNEDQNHHEQPFFYPLGSSSSLHHHHHHHHHQVPSNSSSSSSSISSLSSYLPFLINSQEDQHVAYNNTYHADHLHLSQPLKAKMFVANGGSSACDHMVPKKETRLKLTIRKKDHEDQPHPLHQNPTKPDSDSDKWLMSPKMRLIKKTITNNKQLIDQTNNNNHKESDHYPLNHKTNFDEDHHEDLNFKNVLTRKTTAATTENRYNTINENGYSNNNGVIRVCSDCNTTKTPLWRSGPRGPKSLCNACGIRQRKARRAAMAAAAAAGDQEVAVAPRVQQLPLKKKLQNKKKRSNGGEKYNHSPPMVAKAKKCKIKEEEEKEMEAETVAGDSEISKSTTSSNSSISSNKFCFDDLTIMLSKSSAYQQVFPQDEKEAAVLLMALSYGMVHG

SEQ ID NO: 102 - protein, 352 aa, Arabidopsis thaliana
  misc_feature: Public GI ID no.15236131
MGSNFHYTIDLNEDQNHQPFFASLGSSLHHHLQQQQQQQQHFHHQASSNPSSLMSPSLSYFPFLINSRQDQVYVGYNNNTFHDVLDTHISQPLETKNFVSDGGSSSSDQMVPKKETRLKLTIKKKDNHQDQTDLPQSPIKDMTGTNSLKWISSKVRLMKKKKAIITTSDSSKQHTNNDQSSNLSNSERQNGYNNDCVIRICSDCNTTKTPLWRSGPRGPKSLCNACGIRQRKARRAAMATATATAVSGVSPPVMKKKMQNKNKISNGVYKILSPLPLKVNTCKRMITLEETALAEDLETQSNSTMLSSSDNIYFDDLALLLSKSSAYQQVFPQDEKEAAILLMALSHGMVHG

SEQ ID NO: 103 - DNA, 1371 bp, Arabidopsis thaliana
  misc_feature: Ceres CLONE ID no.18857
gacgagacattgaaaactcccatctaccttttctctttctctctctctccctcccaaagtcgaacctgcaatccctaaatccccaatttgagaccatctccagttcgtcatcgtacgacgactctctctctctctctcctcttctctgtatagtcgaattatattacaagttagatagatagcatgaattggttacctgaagctgaagctgaggatgacttcaaaggtcttctctctggtgatttctttgatgatctcatcaatcaccttgacgtccctcttgatgacattgataccaccaatggtgaaggagattgggttgacaggtttcaagaccttgagcctcctcccatggatatgttccccactttgccttctgacctcacctcttgcggctctggcatggccaaggcccctcgtgtggatattcaaaggaacattcctgctttgaaacagtcctattcttctgaagccttgtccagcactctccaccaatcttcagctcctcctgaaatcaaagtttcgaaactatttcagtctttaagtccagtgtcagttctcgagaacagttatggttctctctcgacccacaacaatggatctcagagattggctttccctgtgaaaggcatgagaagcaagcgcaaacgccccacaacactgagacttagctaccttttcccctctgaaccccgaaagcccgagaagtcaaccccgggcaaacctgaatccgagtgttacttttcttctgagcaacatgccaagaagaaacgcaagatccacctgaccacccgcacggtctcttccactctggaggcaagcaactcggatgggatagtccgcaaatgcactcattgcgagacaaccaagaccccacagtggagggaaggacccagtggaccgaagaccctctgcaatgcttgcggagtgaggttcagatctggtcgcctagttccagaataccgaccagcctcaagccccaccttcatcccagctgtgcattcaaactcacacaggaagatcattgagatgagaaggaaggacgacgaacagtttgatagcagcatgattcgggcagtgatatccagagggtaaagcagttttttttttaaagtttggtctctgatccgcagaaatgggaagtcagtggaggaagaagatggtataaaagtagggtttagtgactgttttcaaatctgatgaagagagtttagtggtttagttcaagttagggatatgaaatattttgttgtttcattatgtgtttttatctttatctcttggctgacatttgtgtatcagaggaaaacaggtgaagccaaagtttgctctctttttttcgctatttcaaatctattctttttattcttc

SEQ ID NO: 104 - protein, 245 aa, Arabidopsis thaliana
  misc_feature: Ceres CLONE ID no.18857
MFPTLPSDLTSCGSGMAKAPRVDIQRNIPALKQSYSSEALSSTLHQSSAPPEIKVSKLFQSLSPVSVLENSYGSLSTHNNGSQRLAFPVKGMRSKRKRPTTLRLSYLFPSEPRKPEKSTPGKPESECYFSSEQHAKKKRKIHLTTRTVSSTLEASNSDGIVRKCTHCETTKTPQWREGPSGPKTLCNACGVRFRSGRLVPEYRPASSPTFIPAVHSNSHRKIIEMRRKDDEQFDSSMIRAVISRG

SEQ ID NO: 105 - DNA, 1672 bp, Panicum virgatum
  misc_feature: Ceres CLONE ID no.1790292
atgatcaccctgggccctttgtaaattgcccctccaactgcagatctcctctccatctccctgcagcttatatgcatcatctatccctgatctcttccttctttccccccagcgcttgctacctctctagctagctagatctctctcctctacatgtctaccatctacatgagccaactgtctactctccctctaatggagcgagagcaggatcaggggctctttccggccttccatgtcacgaaggacccacctatcttattcccgttcatgatcaacaaccctgtggatcagctccaagggcaaagtagctatggagatcaacgcttgaggcatcaagttttggctgaatctactcatcaacaggttaggggccgggatcataattttggtatggatagcatcaaaggatagctatatatcttagcatgcttgatgctcttttagtcttagcagctccatgccatgtttaattagtcttccttttctcttttgtatatatgcagttcactgatcgcatgatgatgatgagcggaggatcagacgtcttccctaggccatcaccgttccggccgactattcaaagcatcgatggtgacatgatccagcggtccgcgtatgatccatacgatatcgagaacaagcgtgccgtcgatggatcgatcagtggctgggcagcggcggcggcaccgccggcgaagatgaagatcatgaggaaggcgatggcgagtgagtatcccgagggcggggcagcgagaaagccaaggagaagagcccaagcacaccaggatgagagccagcagctgcagcagcaggctgctatgggtgtcgttagggtgtgctccgactgcaacaccaccaagacccccttgtggaggagtggtccttgtggccccaagtctctttgcaacgcatgcggcataaggcaaaggaaggcgcgtcgggcgatggcggccgccgccggtgccagcaacggaggcgctccacaggtggccagtgtcgccgcacaggcaaagccggcgtccaagaaggagaagagggcggacgtcgaccagtcgctgccgttcaagaaacggtgcaagatggttgccgtggatcacgctgtcacggcggccaaggagacgccgacagtcgctgctccgtccaaccgggatcaggatctggatcacgtctccagtgacaaggttgtgagtctgcagagcaaggtcgtcgccactcctgatccgccggcgatgtcgttccacgccttccccgccgacgagatcaccgacgccgccatgctgctcatgacactgtcctgtgggctcgtccgcagctagctagcgagctatatacagcttctactagcagctgatagcgagatcactggtagacactgatgagttcgacacgttttgcaactaagtaccctaccccggccctatcatattgcttgtttgatcaagtgttgaggaagagcaagtaagatcaccaggagagatagagtgatcacatgcatggccaaggcaaaataataataagccaaaaactctggttagcctaccaggatttgctataagagtgagttttttaatttgttttaccttttttcccagctgtggctaattattcaagccactatatatatatgctatgcaactttgttcatttgtaaaaaaaaaaaaaaaaaaaa

SEQ ID NO: 106 - protein, 272 aa, Panicum virgatum
  misc_feature: Ceres CLONE ID no.1790292
MQFTDRMMMMSGGSDVFPRPSPFRPTIQSIDGDMIQRSAYDPYDIENKRAVDGSISGWAAAAAPPAKMKIMRKAMASEYPEGGAARKPRRRAQAHQDESQQLQQQAAMGVVRVCSDCNTTKTPLWRSGPCGPKSLCNACGIRQRKARRAMAAAAGASNGGAPQVASVAAQAKPASKKEKRADVDQSLPFKKRCKMVAVDHAVTAAKETPTVAAPSNRDQDLDHVSSDKVVSLQSKVVATPDPPAMSFHAFPADEITDAAMLLMTLSCGLVRS

SEQ ID NO: 107 - DNA, 1776 bp, Panicum virgatum
  misc_feature: Ceres CLONE ID no.2007914
atcgccgctttgcctcttgatcccttcgaattaattgcccgcatcagacggctgccgttctcctcctcgctccgatcccctctccacatctcatcctgatctccactcactcatcatcacctagctagctctactacctagccagcagccatagctccatccggatctcgggcgcgagttcctctcggtcgtcccatgtctgccatctagtatctacatgagccagctctccactgccctccctctcatggagggagatcaccaccaggatcaccaccaccaccaccaccaaggccacttccaggccttcaccctccccaaggatcccccgatcctgttcccctttgtgatcagcagcagcagcagcgccagcgacagcagcctgagctatggatcgccagaaggccatcacttgttcaggcagcagcagcatcaggctatgctggagccccagcatatgatcggcggatcatcgtctgcgagtgtctttgcgacgccgttcccgaccgtggagagcatccgggacgacatgatcgagccgtcctcgttcgatccatacgatatggggaggctgcagatgggtggatcacttgaggctgcaggcagctggacgccgccggccaagatgaggatcacgaggaaggcgaccgccgatcccggtgcggcgaagaagcccaggaggaaggcacaagggtacgaggacatgctgagcatgggtgggctgcctaatctgggtgttattagggtgtgctccgactgcaacaccaccaagacgcccttgtggaggagtggtccttgcggccccaagtcgctctgcaacgcgtgcggcatcaggcagaggaaggcgcggcgggcgatgatggcctctgggggcggctccgggccagtgccggccgacggcgccaaggcagccactgccacgcccagggacgccatggcagcacgagcacactcgaaggtgaagaaggagaagagagtggacgtggaacggtcgctgccgttcaagaaacggtgcaaggtggtccaggatcacggtggcgccgccgccgccgccgcccctcctgccgccgctcacaaggccgtcccccagcccgccgccgaggttgctgatgacgccagcctgagcagcagggaccttgggctcctcagctggagcaggagcccagctcgcccatcttccgctgcttcctgcagtttccgggtgtcgccggccctgcctgtgcagcaggacgagatcacggatgccgccatgttgctcatgacgctttcctgcgggcttgtccggagctgatgatgatctacctctacatgtacacaaggcacgaccagcaccgtaggccttttgcagcagctaggtgcctttgactgttagtgttaccgtcgtcgtctaccttccgcgctgtagtggtttttggcttcttgttcgttgccagtgtaacccgtgtcactgttggagtcatgtgattcgttcgtgtatgagagagatcagagagagaagagatgataggagcgacaggatcacgagggaagtaataaggcaaaatcgtgagattaccactagctagaaatatagctaggatataattgcatgtagtagtgtgaggagtttcagttttgagtaatagttttttctgtgtgctggggctataacatggtcgctggctctgctagcagctagtagcttgctacatgtatttttgttctttgttgttttggttcagtttcttgtactggggctaaaaataatagcaatatgcatggagagaggtcgagcac

SEQ ID NO: 108 - protein, 357 aa, Panicum virgatum
  misc_feature: Ceres CLONE ID no.2007914
MSQLSTALPLMEGDHHQDHHHHHHQGHFQAFTLPKDPPILFPFVISSSSSASDSSLSYGSPEGHHLFRQQQHQAMLEPQHMIGGSSSASVFATPFPTVESIRDDMIEPSSFDPYDMGRLQMGGSLEAAGSWTPPAKMRITRKATADPGAAKKPRRKAQGYEDMLSMGGLPNLGVIRVCSDCNTTKTPLWRSGPCGPKSLCNACGIRQRKARRAMMASGGGSGPVPADGAKAATATPRDAMAARAHSKVKKEKRVDVERSLPFKKRCKVVQDHGGAAAAAAPPAAAHKAVPQPAAEVADDASLSSRDLGLLSWSRSPARPSSAASCSFRVSPALPVQQDEITDAAMLLMTLSCGLVRS

SEQ ID NO: 109 - protein, 347 aa, Oryza sativa subsp. indica
  misc_feature: Public GI ID no.125538648
MSTIYMSQLPATLPLMEGDQDQGLYPAFHRAKDPPILFPFMIDSAVEHQGQIYGDQGLRRQQVLGESNQQFNDHMMMGGSDVFLTPSPFRPTIQSIGSDMIQRSSYDPYDIESNNKQHANGSTSKWMSTPPMKMRIIRKGAATDPEGGAVRKPRRRAQAHQDESQQQLQQALGVVRVCSDCNTTKTPLWRSGPCGPKSLCNACGIRQRKARRAMAAAANGGAAVAPAKSVAAAPVNNKPAAKKEKRAADVDRSLPFKKRCKMVDHVAAAVAATKPTAAGEVVAAAPKDQDHVIVVGGENAAATSMPAQNPISKAAAASPAFFHGLPRDEITDAAMLLMTLSCGLVHS

SEQ ID NO: 110 - protein, 372 aa, Oryza sativa subsp. indica
  misc_feature: Public GI ID no.125555801
MEGEHHQDHHQGHFQAFSLQPKDPPVLFPFVINRRSSSSSPSDSTTLSYGSDHHLTQQQQHQHQAMLEPQHMIGGSSAGIFATPFPTVKSIRDDMIERSQFDPYDTEKLQASCGLAKVVAGGKWSAVPAAKMKITRKMGEPSSGVTGGAATTVAPKKPRRRLAQAYEDHGHGGAMGQAFGVIRVCSDCNTTKTPLWRSGPCGPKSLCNACGIRQRKARRAMMASGLPASPNAAGPKAAAHSGAAAVAAAQPKVKKEKRADVDRSSLPFKKRCKAVQVEDHQTLPAATNAAAAAAMEETAESATVAPPPAPTTRGCTLVDSIGLSWSKTHAAATASCSFRPSPVAPGFAAAVQDEITDAAMLLMTLSCGLVRS

SEQ ID NO: 111 - protein, 353 aa, Oryza sativa subsp. japonica
  misc_feature: Public GI ID no.115445073
MSTIYMSQLPATLPLMEGDQDQGLYPAFHRAKDPPILFPFMIDSAVEHQGQIYGDQGLRRQQVLGESNQQFNDHMMMGGSDVFLTPSPFRPTIQSIGSDMIQRSSYDPYDIESNNKQHANGSTSKWMSTPPMKMRIIRKGAATDPEGGAVRKPRRRAQAHQDESQQQLQQALGVVRVCSDCNTTKTPLWRSGPCGPKSLCNACGIRQRKARRAMAAAANGGAAVAPAKSVAAAPVNNKPAAKKEKRAADVDRSLPFKKRCKMVDHVAAAVAATKPTAAGEVVAAAPKDQDHVIVVGGENAAATSMPAQNPISKAAATAAAAAASPAFFHGLPRDEITDAAMLLMTLSCGLVHS

SEQ ID NO: 112 - protein, 347 aa, Oryza sativa subsp. japonica; identical to SEQ ID NO: 80
  misc_feature: Public GI ID no.115468636

SEQ ID NO: 113 - protein, 375 aa, Oryza sativa subsp. japonica
  misc_feature: Public GI ID no.125597642
MEGEHHHHHQDHHQGHFQAFSLQPKDPPVLFPFVISRRSSSSSPSDSTTLSYGSDHHLTQQQQHQHQAMLEPQNMIGGSSAGIFATPFPTVKSIRDDMIERSQFDPYDTEKLQASCGLAKVVAGGKWSAVPAAKMKITRKMGEPSSGVTGGAATTVAPKKPRRRPAQAYEDHGHGGAMGQAFGVIRVCSDCNTTKTPLWRSGPCGPKSLCNACGIRQRKARRAMMASGLPASPNAAGPKAAAHSGAAAVAAAQPKVKKEKRADVDRSSLPFKKRCKVVQVEDHQTLPAATNAAAAAAMEETAESATVAPPPAPTTRGGTLVDSIGLSWSKTHAAATASCSFRPSPVAPGFAAAVQDEITDAAMLLMTLSCGLVRS

SEQ ID NO: 114 - protein, 354 aa, Oryza sativa subsp. japonica
  misc_feature: Public GI ID no.125581335
MSTIYMSQLPATLPLMEGDQDQGLYPAFHRAKDPPILFPFMIDSAVEHQGQIYGDQGLRRQQVLGESNQQFNDHMMMGGSDVFLTPSPFRPTIQSIGSDMIQRSSYDPYDIESNNKQHANGSTSKWMSTPPMKMRIIRKGAATDPEGGAVRKPRRRAQAHQDESQQQLQQALGVVRVCSDCNTTKTPLWRSGPCGPKSLCNACGIRQRKARRAMAAAANGRSGRWRRQRGVAAAPVNNKPAAKKEKRAADVDRSLPFKKRCKMVDHVAAAVAATKPTAAGEVVAAAPKDQDHVIVVGGENAAATSMPAQNPISKAAATAAAAAASPAFFHGLPRDEITDAAMLLMTLSCGLVHS

SEQ ID NO: 115 - DNA, 1698 bp, Triticum aestivum
  misc_feature: Ceres CLONE ID no.912192
atctctctcgatcggctctctgcaaactgcacatcacagtgctagctagctagctcctcccctccccatctcatcctgatctccaattatctgcgctccctagctcgctagatctcctctagatctcatcatgtctaccatctacatgagccagctctctactgctttccctctaatggaggaagatcatcaccaggatcaccatcaggggcacttccaggccttcaccctgcccaaggatccccccatattgttcccttttgtgatcagcaacagcggccctagcgacaacagcagtactctcagctacggatcagaccagcacttgatgcagcaccatgccatgctagatcagccccaacatatgatcggtgaatcatccagtgtgttctcgacgccgttcccgaccgtcgagagcatccgcgacgacatgatcgagcgatcctactcgtacgatccatatgatatggagaagctgcaggccaccagcggatcgctcaagatcggcaagtggacggcgcctgctcccgcagccaagatgaggatcacgaggaagacgagcgatcctggtgtcaagaagccgaggaagagggcgcaggcatacgaggatcatggccacatgggcggcatgaaccaagctctgggtgttattaggacatgctctgattgcaacaccaccaagacccccttgtggaggagtggtccttgtggccccaagtcgctttgcaacgcgtgcggcatcaggcagcggaaggcgcgccgggcgatgatggccaccggagcggcgcccgccacggatgttggtgccaccaaggcggccgcgccgggtgacgacgccgtgacggtgcacccaccgaaggtgaagaaggagaagagagctgccgacgtcgaccggtctctgccgttcaagaaacggtgcaaggtggtccaggatcacactgccacaaacgccgctgcttccaccaccgtcgaggctgccacagagccaccggtcgtcgtcaccaccaccaccaccaccactgctgctgctgctgcaccggcgagggatctcgtcgacaccatcggggccaactggagcaagagccctactgtcgccgcagctgcttgcttccggccatcgccggcgacgttcgcggtgccggtgccggtgcaggacgagatcacggacgccgccatgctgctgatgacgcttcctgcgagcttgtccggagctgatcgatccacacatggaacactcgagagactctgatctatccaccacggtacccccccgggcttttctgccttagtccgttagacagtcggtacgtactgtccgtgctgccgcgtttttggtttcttgttcttgttgctagtgtaaccctgcgctactgttaaagtcttgtgattcgtttgtgtgtaagataagatggtgagaaagagagagatctagagaaagagaggtagaaacaaggagagatcagaggagagaagaggataaaaataaaaaaacaacgtgagctagctagctaggatttgcaaacactgtgagatcgagtgagttgagtttgagtagctagctttttgttgcchcggytaataaccmagccaatggytactatatagcatgtatgttttgttctttgttttgctcgaaccatttgtactgtgactcgtmataataaataccsaaagrccggcccmaaaaaaaaaaaaaaa

SEQ ID NO: 116 - protein, 372 aa, Triticum aestivum
  misc_feature: Ceres CLONE ID no.912192
MSTIYMSQLSTAFPLMEEDHHQDHHQGHFQAFTLPKDPPILFPFVISNSGPSDNSSTLSYGSDQHLMQHHAMLDQPQHMIGESSSVFSTPFPTVESIRDDMIERSYSYDPYDMEKLQATSGSLKIGKWTAPAPAAKMRITRKTSDPGVKKPRKRAQAYEDHGHMGGMNQALGVIRTCSDCNTTKTPLWRSGPCGPKSLCNACGIRQRKARRAMMATGAAPATDVGATKAAAPGDDAVTVHPPKVKKEKRAADVDRSLPFKKRCKVVQDHTATNAAASTTVEAATEPPVVVTTTTTTTAAAAAPARDLVDTIGANWSKSPTVAAAACFRPSPATFAVPVPVQDEITDAAMLLMTLPASLSGADRSTHGTLERL

SEQ ID NO: 117 - protein, 398 aa, Arabidopsis thaliana
  misc_feature: Public GI ID no.17473547
MDSNFHYSIDLNEDQNHHEQPFFYPLGSSSSLHHHHHHHHHQVPSNSSSSSSSISSLSSYLPFLINSQEDQHVAYNNTYHADHLHLSQPLKAKMFVANGGSSACDHMVPKKETRLKLTIRKKDHEDQPHPLHQNPTKPDSDSDKWLMSPKMRLIKKTITNNKQLIDQTNNNNHKESDHYPLNHKTNFDEDHHEDLNFKNVLTRKTTAATTENRYNTINENGYSNNNGVIRVCSDCNTTKTPLWRSGPRGPKSLCNACGIRQRKARRAAMAAAAAAGDQEVAVAPRVQQLPLKKNLQNKKKRSNGGEKYNHSPPMVAKAKKCKIKEEEEKEMEAETVAGDSEISKSTTSSNSSISSNKFCFDDLTIMLSKSSAYQQVFPQDEKEAAVLLMALSYGMVHG

SEQ ID NO: 118 - DNA, 1290 bp, Gossypium hirsutum
  misc_feature: Ceres CLONE ID no.1847255
ggacgaagccaacaccgtcgcttcgattgctccatctttccccatgacttctagtaattgttattcatctatttatccttttcctattgacctaaacgaagatgatcaacaccaacaccaccagcttttcaacttaatatctcaaccttcttcttcttcttcttcatcatcttcttcttcgtcgccttctacttcacttacttgtcctttttccttcagcccaactgttcaacaccagcaagcagctttctatcagagcctaccccagcagttccatgatgatcaacaagatcaggaaaaaatacatattccacaagatggaccattgaggagtgactgtgagctcaggttatctatatggaaaaaggaagaaagggttgaaactcatcatcaaagtcatgattctgctaagtggatgccttcaaagatgaggatgatgaggaagatgatgagctccgatcacaccgatttatcaaatagccctacaccgaaatccgaagatcaccaggagcagaaacaaccatcatcttctccagacaacaacaacagcacaattagggtttgtgctgattgcaacactactaagactcctctctggaggagtggaccccggggccccaagtccctttgcaacgcctgtggaattcgtcaaagaaaagcaagaagggctgcagcggtggcggcggcggcggcaagcagcgttgttgcagctgagacaccgccaagtatgagaagtgaagttcaacttaaggcgaagagatcaagcaacaatggtgttccacatttgaagaacaaaaaatgcaaacataattctcaatctcaaagtagaaagaagctgtgtttcgaagatttaaggataatattgagcaagaattcagcttttcatggagtttttcctcaggacgagaaagaagccgcgatcctactcatggccttatcgtatggtcttgttcatggttgaaaattctccccaccaccttagccttatatattgttgttttctaggtttaaataattagtgttgggagtgtagtagctaactaaggtcataaacgtttgaaactagaattgtcattgagtgtgaagaaaaagtaatttgttaaaataaagaaaaagagatttgaaatgcaaaatgcatgtagaactttagagctttgattatcaggtgaatttgaacaggttggtaaccataattatggagctcttgttcttctacgtcttattctatatatattatgtcgtgaacttaataaaattctatgctttgattaaaaaaaaaaaaaaaaaaaa

SEQ ID NO: 119 - protein, 305 aa, Gossypium hirsutum
  misc_feature: Ceres CLONE ID no.1847255
MTSSNCYSSIYPFPIDLNEDDQHQHHQLFNLISQPSSSSSSSSSSSSPSTSLTCPFSFSPTVQHQQAAFYQSLPQQFHDDQQDQEKIHIPQDGPLRSDCELRLSIWKKEERVETHHQSHDSAKWMPSKMRMMRKMMSSDHTDLSNSPTPKSEDHQEQKQPSSSPDNNNSTIRVCADCNTTKTPLWRSGPRGPKSLCNACGIRQRKARRAAAVAAAAASSVVAAETPPSMRSEVQLKAKRSSNNGVPHLKNKKCKHNSQSQSRKKLCFEDLRIILSKNSAFHGVFPQDEKEAAILLMALSYGLVHG

SEQ ID NO: 120 - DNA, 1134 bp, Sorghum bicolor
  misc_feature: Ceres ANNOT ID no.6111325
atgtctaccatctacatgagccaactgtctactctccctctaatggagatggagggagagcaggatcaggatcagggactctttcggccggcgcccttccatgtcgcaaaggaccctcccatcttgttcccgttcatgatcaccaatcctgtggatcagctccaagggcaaagcagcagctatggagatcagcacttgaggcagcatgttttgtctgaatctactcatcaacagttcaccgatcggatgatgatgagcggatcagaagtcttccctactatgccatcgccgttccggcagactatccaaagcatcgatggtgacatgatccagcgatctgcgtatgatccatatgatacagagaataataagcacaccgaaggattgcgcagcggcggcggcggcggcggctggacagtgtcgtcgtcaccgccagcgaagatgaagatcatgcggaggaaggcaacgagcgagtatcccgagggcggtggtgggggagcaagaaagccaaggagaagaccacaagcgcaccaccaagatgctgagagccagctactccagcagcagcagcaggctatgggtggtgtcgttagagtgtgctccgactgcaacaccacaaagacccccttgtggaggagtggtccttgtggccccaagtctctttgcaacgcatgtggcataaggcaaaggaaggcgaggcgggcgatggcggcagccgcggcagctgctgctgccagcagcaacggaggcgctccacaagcggccaccgagcagcagccgaagccggccaagaaggagaagaggtcggacatcgtcgaccggtcgtcgctgccgttcaagaagcggtgcaagcagacgatgccactgaagcgacgccgccgccgccgccgccaggaaaccgctgctctgaccaaggatcaggcggatgtggatcacgtctccagtgacaagaagcaggttaatggccatggcgcgacgagtctggagagcagcaagatcgtcgccactcctcctccccaggcgatgtcgtcgtttcatgccttcccggctgacgagatcactgacgccgccatgctgctcatgaccctgtcctgtgggcacgctcgtctgcagctagccatggcttctagctag

SEQ ID NO: 121 - protein, 377 aa, Sorghum bicolor
  misc_feature: Ceres ANNOT ID no.6111325
MSTIYMSQLSTLPLMEMEGEQDQDQGLFRPAPFHVAKDPPILFPFMITNPVDQLQGQSSSYGDQHLRQHVLSESTHQQFTDRMMMSGSEVFPTMPSPFRQTIQSIDGDMIQRSAYDPYDTENNKHTEGLRSGGGGGGWTVSSSPPAKMKIMRRKATSEYPEGGGGGARKPRRRPQAHHQDAESQLLQQQQQAMGGVVRVCSDCNTTKTPLWRSGPCGPKSLCNACGIRQRKARRAMAAAAAAAAASSNGGAPQAATEQQPKPAKKEKRSDIVDRSSLPFKKRCKQTMPLKRRRRRRRQETAALTKDQADVDHVSSDKKQVNGHGATSLESSKIVATPPPQAMSSFHAFPADEITDAAMLLMTLSCGHARLQLAMASS

SEQ ID NO: 122 - DNA, 1161 bp, Sorghum bicolor
  misc_feature: Ceres ANNOT ID no.6065280
atgtctgccatctacatgagccagctctccactgctctccctctcatggagggagatcaccaccaccaccaccaccaccaccaccaaggccacttccaagccttcacgctgccaaaggatcccccaatccttttcccctttgtgatcagcaatagtagcgccagcgaaagcagtctgagctatggatcggcagatcatcacttgttgaggcagcatcgtcaaactatgctcgagccccaacatatgattggtggatcgtcatcagctgcgagtagtgtctttgcgacgccgttcccgactgtggagagcatccgtgacgacatgatcgagcctgcctcgtacgatccatacgatatggggaagctgcaccaggtggtcggtggcgggtcgtcgatggatgcttgcagctggacgccgccggcggccaagatgaggatcacgaggaaggccactgccgccgatcccagtggtgccgggaagaagccgaggagaagggcgcatcaggcagcagggtacgacgccgacatcaacatgagcggccaaccaaatttgggtgttattagggtgtgctccgactgcaacaccaccaagacgcccttgtggaggagtggtccttgcggccccaagtcgctctgcaacgcgtgcggtatcaggcagcggaaggcacggcgggcgatgatggccgccgcgtctacctccgggccagcagcagtgcccgccaccgacagcgataaggcctcgccgagcaacgccgccggggccgcagcagcacaccccaaggtgaagaaggagaagagatcagtggacgtggaccggtcgctgccgttcaagaaacggtgcaaggtggtccaggtccagcaggatcacgctgcggccgtcgcagctcctgccgcggccactgacaggccggctgtcgtcgtgcagcaggcggccaccgccgccgaggttggtgacgacgacgcctgtccgagcagggacctgcttgtcgacgacattggtgggctcatcagctggagcaggagtccgccggcggctcccgcatctgcagatgctgcctcctgcagtttccgggcgtcgccggcgttgccggtgcagcaggacgagatcacggacgctgccatgctgctcatgacgctgtcctgcgggcttgtccgaagctga

SEQ ID NO: 123 - protein, 386 aa, Sorghum bicolor
  misc_feature: Ceres ANNOT ID no.6065280
MSAIYMSQLSTALPLMEGDHHHHHHHHHQGHFQAFTLPKDPPILFPFVISNSSASESSLSYGSADHHLLRQHRQTMLEPQHMIGGSSSAASSVFATPFPTVESIRDDMIEPASYDPYDMGKLHQVVGGGSSMDACSWTPPAAKMRITRKATAADPSGAGKKPRRRAHQAAGYDADINMSGQPNLGVIRVCSDCNTTKTPLWRSGPCGPKSLCNACGIRQRKARRAMMAAASTSGPAAVPATDSDKASPSNAAGAAAAHPKVKKEKRSVDVDRSLPFKKRCKVVQVQQDHAAAVAAPAAATDRPAVVVQQAATAAEVGDDDACPSRDLLVDDIGGLISWSRSPPAAPASADAASCSFRASPALPVQQDEITDAAMLLMTLSCGLVRS

SEQ ID NO: 124 - protein, 304 aa, Sorghum bicolor
  misc_feature: Ceres ANNOT ID no.6074550
MVEKGCERCKEWQEHYYWEHMDVSKIRFFKRMTGDFAHRTSIPEKFVKKFNGQITEGVDMKTPSGETWHVGVAKNQDELFFVSGWEDFVKAHELKENDLLLFTCRGSSSFQVVIFEGSGCEKVSSVFGNRFGKQPESPNSNSDYDVKQQGTREEDSDSDNEYTDFKYYYSRTANRLSNDEKSKIISLASIQPDNPAFITVLQMTNVRPRNNSLTIPRQFAADHLEERSQEIILRRPNRKDKWYVRYYYTTAIRSLKSYHWSKFVRENKLREGDICVFELMKGAKRVTMSVHVIKKVENRFVLLG

SEQ ID NO: 125 - DNA, 1470 bp, Glycine max
  misc_feature: Ceres CLONE ID no.706980
gaaaaacaaacacagatagaaagaaaagaaatttcaccgtaccagagaacaaggaaggatagaagaaagtgaaagaaaatggcgttgtacatggacgaagaagagatttggaagtgtccgaagcatccatcaaagagaagaagaagcggaatttgccctacttgccttcgagaccgtctcgtaactctttgccctgactgtgctaacgtgcgcccctgttcctgctacgccaccagctcctcctcctcttcctcgtcttcctcctccttatcgcgtttttctggcgccggcgatggcgtcggttccgtcggccgcgtccacaacctcatcgagcaagagccagggctccgccgctcgcgctccatggcgattccgtttctcaggtcacggtcgcggttctctggcggcggagatagggtttcggatctcgacagcacgagggactcgccggcaatgaacggaagccggtcggcgaggtcgttctggtcaatgttcaagtcgcagaagagcagtagacacggcggcggaggaggcggcgcgccggagcaagagtgggaggcgaaaaagatattggcggaggaacgagacggcgatggtggcataaatccgatgatggtgcggtcaaggtcggtggcagtgacggcggtaacggtggtttccggcgacggagaattgagaccgcggacgaaagggagagggtggttcttcccaagtccgatgaaggctttccggcaatccaaggtctctaaagtgattcaggaagggtctcctttgtatagaggttgaaattttgtagattaaaaaaattataataataataacaacaaatactttatataaaaaaaaaaaaaaaaagcttctcgtgccggcttctcgtgccgttttttttttttttttttagtaaaagattctcaactcaattctctgcattacaaatgaactttcatgttttcttattattgatttaatttctatcttcaaaatgataaataatagctaatttgccaaacagtaaacaatcacggaaacaaatccaatgatatcacctgtaacagctgaatacactgcctggagaccaaattttgccaatattgataaccagcgattcttcccacttttgctaccagagctagcaacagcacctggaggcgcaccattatcctcaccctttatcattttaaccatgattgtatcaaaaacgctagaaagattgccccggaaattgtcaacatcttctttttcagcatccacactgacctcaagccattctaaggcacgatcaactgcatcattgatcttttccttgtcttccggagaaagcttcaaactaatccccttatgctttatagcattccttatgttgtttcagctgtgtgtcaacaagtcatgtaagagacttcactatgttgttttctctttcgtggctttaataaattatgcgcccttgttccattaaaaaaaaaaaaaaaa

SEQ ID NO: 126 - DNA, 1117 bp, Panicum virgatum
  misc_feature: Ceres CLONE ID no.1822715
cttttccaacagcgacgcaacgagctcgtaacgcccgtaccctcgcacgcgcgcagagagagagagagagagagagagagagagagcaagtgagcagagaagaagaggaaggagcgagaagcctgggaagggagagcgagcgcaaccgaatcgatatgaagccggagaaggcagcggtggccgtggcagccggcggcggcggcgacgagtggcggtgccggaagcacccggccgcgccgagatccggcggtggggtgtgcccgtactgcctccgcgaccggctcctccgcctgtgccccaactgcgcacgcgtgcggccctgcccctgcgccgcgtcgtgcgcctccccgtcctcctcctcggcgtccggcgacgcggtgggccgcgtgcacagcctcatcgagcgggagcaccggatcgcgcgctcgcggtccgtggccgcgggctcctccgccgcgttcgtcgccgccgtgggtgccccctccgcggcggggcccacctccgggggcggcaggcggaaggcgcgcgtctgggggtggccgccgttctggaagcccgcgtcgcgggacggggacgcggggatgggggaggacgaggaggaggggctgggcctgccgcgctcgagctccgtgtccgcgacggccgtggagaccaagaccgcggcggcggcggcgcgcgcgaggtgggggtggcacttcccgagcccgctgaaggcgttccggcaccggaggtcgtcggcgagcgtggccgggcgggcgtgagcgccaaggtacaccgcccccttctggagatggccggaaaagcgattcgcgggtgggggtcatgtggccggggatggttaattattaattttaactgtataaggcggtggattagaggaggagcgttggatctcctttttctccttttgttgtggcgtgccgctaattccatgattaagttagacggggatgcgaaagcctgcgatctgggcgggatgtgtgcatatgctagtgggtgatctcctcatggcttcccttcaccttttgtagtgtgcttgtacccgactctgtaacacaccaaattggatattttaagctctccaatttgttctggctaaaaaaaaaaaaaaaaaaaa

SEQ ID NO: 127 - protein, 201 aa, Arabidopsis thaliana
  misc_feature: Public GI ID no.15226927
MQREKTQTNLRLDEGGGSVWQKCSRHEGRFVCVAGVCPYCLHERLSSLCPDCAHDLPCSCTPRASVSSRGEGDVSFARIGSVGRVANLIECEPAFRRSTSITGPYFWSAKPEPILETEKDLKPGRGRWLWRLFRGNREEETKIKAAKVMKKSNSVAGEVLFSPTPVTSKGNGWYFPSPIKVFRQSRVSKMIFQQRSPLYRG

SEQ ID NO: 128 - protein, 201 aa, Arabidopsis thaliana
  misc_feature: Public GI ID no.28207138
MQREKTQTNLRLDEGGGSVWQKCSRHEGRFVCVAGVCPYCLHERLSSLCPDCAHDLPCSCTPRASVSSRGEGDVSFARIGSVGRVANLIECEPAFRRSTSITGPYFWSAKPEPILETEKDLKPXRGRWLWRLFRGNREEETKIKAAKVMKKSNSVAGEVLFSPTPVTSKGNGWYFPSPIKVFRQSRVSKMIFQQRSPLYRG

SEQ ID NO: 129 - protein, 198 aa, Oryza sativa subsp. indica
  misc_feature: Public GI ID no.125540748
MKAEKAAVAVGGGGGGDEWRCRKHPVARSGGGVCPHCLRDRLLRLCPNCAHVRPCPCTCASPSSSSSASGEAVGRVHTLIEREHRIARSRSVAASSSLAAASTASATAGAVGGRRKARVWGWPPFWKPAAKDGVAAAAEEDDEEEEGMGLARSSSVSATVVEAKAAAAAAKARWGWHFPSPLKAFRHRRSSASMPERG

SEQ ID NO: 130 - protein, 283 aa, Oryza sativa subsp. indica
  misc_feature: Public GI ID no.125549540
MAGDEWRCRKHPAPPCGGVCPYCLRDRLLRLCPECAHARPCPCAASSSSPSSSSSSAASGSAAVVGRVYSLIERERRMGLRSRSIAAGGGGGGRGIVVRDERPKSRAFGWVSFRKATTSDRVVEVDDGAALARSSSVSATAVETRAPPKSRGWGRFIPGSIKALRHRKSRAAGDCREGVSASLEGKNSKMRMIWASWRSVLHYIVLDQILPLVLRNLRPYKVWKKKTEFGAADARDVPQTTSRKIAERCPAVKLRNREDKIICKEVSTTLATTLIPSPMMVRA

SEQ ID NO: 131 - protein, 198 aa, Oryza sativa subsp. japonica; identical to SEQ ID NO: 92
  misc_feature: Public GI ID no.115448039

SEQ ID NO: 132 - protein, 230 aa, Oryza sativa subsp. japonica
  misc_feature: Public GI ID no.58532121
MAGDEWRCRKHPAPPCGGVCPYCLRDRLLRLCPECAHARPCPCAASSSSPSSSSSSAASGSAAVVGRVYSLIERERRMGLRSRSVAAGGGGGGRGIVVRDERPKSRAFGWVSFRKATSDRVVEVDDGAALARSSSVSATAVETRAPPKSRGWGRFIPGSIKALRHRKSRAAGDCREGVRARSRAKDVVPHSIAGALCYAQCDANPIWLGWVHRRILVMGNGSLSRTCPVV

SEQ ID NO: 133 - protein, 179 aa, Oryza sativa subsp. japonica
  misc_feature: Public GI ID no.115460224
MAGDEWRCRKHPAPPCGGVCPYCLRDRLLRLCPECAHARPCPCAASSSSPSSSSSSAASGSAAVVGRVYSLIERERRMGLRSRSVAAGGGGGGRGIVVRDERPKSRAFGWVSFRKATSDRVVEVDDGAALARSSSVSATAVETRAPPKSRGWGRFIPGSIKALRHRKSRAAGDCREGVR

SEQ ID NO: 134 - protein, 221 aa, Oryza sativa subsp. japonica
  misc_feature: Public GI ID no.125583319
MKAEKAAVAVGGGGGGDEWRCRKHPVARSGGGVCPHCLRDRLLRLCPNCAHVRPCPLHVRGPRPPPRRRPAKRVGRVHTLIEREHRIARSRSVAASSSLAAASTASATAGAVGGRRKARVWGWPPFWEARGQGRRCCCGGGGRGGGGGDGAREVELGLRDGSGGKSGGRGGEGAVGVALPEPAQGVPAPEIVGEHAGARMSEMARHGRKKRSVGMCAALED

SEQ ID NO: 135 - DNA, 600 bp, Sorghum bicolor
  misc_feature: Ceres ANNOT ID no.6086011
atgaaggcggagaaggcagcggtggccccggccggcgccggcggcgacgaatggaggtgccggaagcaccctgccgcgagatccggcggcggggtgtgcccgtactgcctgcgggaccgcctcctccggctctgccccaactgcgcgcacgtgcggccctgcgcctgcgccgcgtccccgtcgtcgtcctccacctcggcctccggcgacgcggtcggccgcgtccacagcctcttcgagcgggagcaccggatcgcgcgctccaggtccgtggccacgggctcctccgccatcttcgccgccgcggtcgccgcgccctccgctgtggcctcgggcaggcggaaggcccgggtctggggttggccgccgttctggaagcccgcggcgcgggaggggggcgcggagctggagctggaggtggaggacgaggacgaggggctggggctgccgcgctccagctccgtgtccgccacggcggtggaggcgaagaccgcggccgcggctagggccgcgcgggcgcggtgggggtggcacttcccgagcccactcaaggcgttccggcaccgcaggtcgtcggcgagcgtggcggggcggccgtga

SEQ ID NO: 136 - protein, 199 aa, Sorghum bicolor
  misc_feature: Ceres ANNOT ID no.6086011
MKAEKAAVAPAGAGGDEWRCRKHPAARSGGGVCPYCLRDRLLRLCPNCAHVRPCACAASPSSSSTSASGDAVGRVHSLFEREHRIARSRSVATGSSAIFAAAVAAPSAVASGRRKARVWGWPPFWKPAAREGGAELELEVEDEDEGLGLPRSSSVSATAVEAKTAAAARAARARWGWHFPSPLKAFRHRRSSASVAGRP

SEQ ID NO: 137 - DNA, 849 bp, Sorghum bicolor
  misc_feature: Ceres ANNOT ID no.6007163
atggacgattggcgctgccggaggcacccgccgctgacgctgcccggcggcggcgtgtgcccgcactgcctccgcgaccgcctcctccgcctgtgccccgactgcgcctgcccgcgtccctgcgcctgctccccttcctccccttcgtcctcgtcctccggcgccagcgcgatcggccgggtccacagcctcatcgagcgggagcaccgggtcgcgcgctcgcgctccgtcgccgcgcacggatccgtcggcgtcggcggcgaccagcggcggcccaagtccggggtgtggggctgggtgtcgttccgcaagcctccaccgccaccgccaccgccggccgcggccgcggggtgcagggacgtggagcaggagtacgacgacgcggtggcattggcgaggtccaggtcggtgtcgtcgatggcggaagcggcagtcccggaggccaaggggccgcctaataaggcggcgcggtgggggaagctcatcccagggaagatcaaggcgctgcggaaccggaagccccgccccgccggcgactgccgcggcggcggtccgggtcccccgcgacccagaaccccacggccacctcgacgccgccgaagagcgcgacggcggcgagcaccccgtagtaggcgcagagcagcagcgtggtaccgccgccgcggtggaacacgccgccggccggcggggatgacctggaggagagagcgaggaggagcgcggccgcggccttggcggagccgccggcgatcagggcacgccgtagcttcgcgccgtcgtgcccggccgccgtggcgtccgccatctgcgccgcgacctccgccgccggcgccgtggcgggcatgctga

SEQ ID NO: 138 - protein, 282 aa, Sorghum bicolor
  misc_feature: Ceres ANNOT ID no.6007163
MDDWRCRRHPPLTLPGGGVCPHCLRDRLLRLCPDCACPRPCACSPSSPSSSSSGASAIGRVHSLIEREHRVARSRSVAAHGSVGVGGDQRRPKSGVWGWVSFRKPPPPPPPPAAAAGCRDVEQEYDDAVALARSRSVSSMAEAAVPEAKGPPNKAARWGKLIPGKIKALRNRKPRPAGDCRGGGPGPPRPRTPRPPRRRRRARRRRAPRSRRRAAAWYRRRGGTRRRPAGMTWRRERGGARPRPWRSRRRSGHAVASRRRARPPWRPPSAPRPPPPAPWRAC

SEQ ID NO: 139 - DNA, 582 bp, Brassica napus
  misc_feature: Ceres CLONE ID no.1090611
agattttgatcgtagctacattactctttcccatggctgcccctgatgaggaccaacggtttattaagcctttcatctcacataagtcagctaaatcattggcgattccgctggccttcaacgaatactttccggatccattgccaaatacagtggagctcctagactactatgggagatcctggacgattaggatgaagaagagaggagagacggtgtttttaactgttggttgggaaaactttgtaaaggataacgagcttgaagacggtaagatgatggaattcatctatgactgcgaccggaccttttatgttgtcatattcggtcatggcggggttagcgagcttagagtctttcctcaagccgtagtagatgttggtgactatgcaacaggcgaagaagaaggagaggaagaggaagaggaagagaagaacaagagtaactgaaacttgagtctgtatcatccacgtagggagaaacaaatgctgcttttgaagttagtttattagtttatttatttaaaagtcttatgtaacaagatgtttaatctaatgaacgaacacactttgttgtcttgct

SEQ ID NO: 140 - protein, 331 aa, Musa balbisiana
  misc_feature: Public GI ID no.102139987
MRTSTGLLYGCFTTIVSWKRIPPKFLKHISSVGSKKAILQGPSGSKWHVELGKDMKDTFLTTGWPKFVKDHSLREYEFLVFQYDGDMRFTVLIFDTSACEREDAFAVRPRRKPRQCEGKRKRGRPPKHSPEVGCIIKSEAIEHGMSLELLKDDVQQPNLLQIQPFQQAESDLLYPAPACDVKVKREESELLINILRNTTKRSKGGRISTRRPLTEKERLRAEEAANSFTSAFPYIVMRMAPSQVYRPYMHLPPKRSNLVLRDPCGKSWIVTYIPSLRCQISRGWSVFARTNKLEEGDYCVLELIGPIELRVHIFRAVEVEETLQIAMTPTT

SEQ ID NO: 141 - protein, 283 aa, Arabidopsis thaliana
  misc_feature: Public GI ID no.6729482
MADDSELYPRFFKVFLVESASESLMIPLPFMAFLADPLPKTVKLQGLGGKLWTVSLKKISGAAYLTRGWPKFAEEHELKNGEFMTFVYDGHRTFEVSVFDRWGSKEVRAEIQAIPLSDSDSDSVVEDEKDSTDVVEDDDDEDEDEDEDDDGSFDEDEEISQSLYPIDEETATDAAGNLDVEALTNPHFPTTLKNRIYELLIPANVVKDNNLEFGSSIKYIDGEGTLVGLRGKWADKRVCFKGWDRICRRNRLKKHQDTVECELLHDDQKMVHSIRVHVLRRDA

SEQ ID NO: 142 - protein, 286 aa, Arabidopsis thaliana
  misc_feature: Public GI ID no.18409814
MADDSELYPRFFKVFLVESASESLMIPLPFMAFLADPLPKTVKLQGLGGKLWTVSLKKISGAAYLTRGWPKFAEEHELKNGEFMTFVYDGHRTFEVSVFDRWGSKEVRAEIQAIPLSDSDSDSVVEDEKDSTDVVEDDDDEDEDEDEDDDGSFDEDEEISQSLYPIDEETATDAAVFEGNLDVEALTNPHFPTTLKNRIYELLIPANVVKDNNLEFGSSIKYIDGEGTLVGLRGKWADKRVCFKGWDRICRRNRLKKHQDTVECELLHDDQKMVHSIRVHVLRRDA

SEQ ID NO: 143 - DNA, 1174 bp, Arabidopsis thaliana
  misc_feature: Ceres CLONE ID no.36830
aagaaacccatttcgattctctctctttctctcgtttccaacatcattccatggctgacgatagcgaattgtatcctcgtttcttcaaagtgttcctcgttgaatcggcttctgaatcgttgatgattccgttgccttttatggcatttcttgctgatccattgccaaagacagtgaagctccaaggtcttggaggaaaactttggactgtgagcttgaagaaaataagcggagctgcgtatcttactagaggatggccaaaattcgcagaggaacatgagctgaagaacggagagttcatgacatttgtgtatgatggtcatcgtacctttgaagtgagtgtctttgatcgttggggtagcaaagaggtcagagctgaaatacaagccataccactttctgattctgattctgattctgttgtggaagacgaaaaagactcaactgatgttgttgaagatgatgatgatgaagatgaagatgaagatgaagatgatgatggtagctttgatgaagatgaagaaatcagccagagtctttatccgattgacgaagagactgcaaccgatgctgcagtttttgaagggaacttggacgtggaagctttaactaatccacactttccgactacgctcaagaacaggatttatgaactgttgatcccagccaatgtggtgaaggataataaccttgaatttggttcatccatcaagtacatcgatggagaaggaaccttagtagggctaagaggaaaatgggctgataagagggtttgcttcaaaggatgggacaggatttgcagaaggaacaggctcaaaaagcaccaagatactgtcgaatgcgagcttcttcatgatgaccagaagatggttcattccatccgagtccatgtcttgcgtagagatgcttgatcctctcaccattcaccaccaccaccatcactctcagtctatcactatcgccgttctcatcatcgcgatcactattatcattatcatcagtactttgacatgacttcttattatggcttttgtttgtttaggagtgttcttatgtttcttcttaggctttatcttagtagtaccttcttcatccccaagaaaacttatgaacttattgctgtttcttgttatatttgatatgctagttataaaatgcatctatggctttttat

SEQ ID NO: 144 - protein, 286 aa, Arabidopsis thaliana; identical to SEQ ID NO: 142
  misc_feature: Ceres CLONE ID no.36830

SEQ ID NO: 145 - DNA, 675 bp, Brassica napus
  misc_feature: Ceres CLONE ID no.1078253
aaaacaaaaaaatttctctccccgtttctgtacacaaggatttaaactgtctctctttactttctcttaaatctctagattttgatcatagctacattaaagtctcactttactctttcccatggctgccattgatgaggaccaacgttttattaagcctttcctctcacataagtcagctaaatcgttggcgattccgttggccttcaacgaatactttccggatccattgccaaatacagtggagctcctagactactatggaagatcctggacgattaggatgaagaaaagaggagagacggtgttcttaactgttggttgggaaaactttgtaaaggataacgagcttgaagacgggaagatgatggaattcatctatgactgcgaccggaccttttatgttgtcatatttggtcatggcggggttagcgagcttagagtcttccctcaagccgtagtagatgttggtgactatgcaaccggtgaagaagaggaagaggaagagaagaacaagagtaactgaaacttgagtatgtatcatccacgtaggtgggaagcaaatgctgctttttaaagtttatttatttgagtctattagtatatcagtttatttatttgagtctatgtaacaagatgtttaatctaatgaacaccaaccctttattgtctgcc

SEQ ID NO: 146 - protein, 134 aa, Brassica napus
  misc_feature: Ceres CLONE ID no.1078253
MAAIDEDQRFIKPFLSHKSAKSLAIPLAFNEYFPDPLPNTVELLDYYGRSWTIRMKKRGETVFLTVGWENFVKDNELEDGKMMEFIYDCDRTFYVVIFGHGGVSELRVFPQAVVDVGDYATGEEEEEEEKNKSN

SEQ ID NO: 147 - DNA, 1785 bp, Sorghum bicolor
  misc_feature: Ceres ANNOT ID no.6053768
atggcgccgctgcgaatatcgtcctctgaagacgggaagtgggaaaggcgtccaactcctgctcgcacgtccgggctgcttagccgcctctccaaatttcgcattcgtcgccgcccctccaaatttcacggattcatattacacaacggtaagattttgacaacgaaagggattcgacagctagtttttatcgcctcggaaaggaattccgccgctatttttatcgcatcattaagtgtgaagagcaagaacacactcaaaaacaccctaaccacatggagaggaatccagccagctctcctgccagatggtgactatctcatctcagagaaacggacgacgatgaggggtcgtcccaagaagcagcaggggaccacgagctcggccgccgcagcggtgaaggagagggaggcggcgagggggaggccgagaagcaaggggaaggagacggagatgggtggagagggtcaggagccagcgacggagaagcagaagaagacccaggggaaggggatggagaaggagaaggagaaggagaagaacacggcggtgagggaagacggcgagcaggacgacaacaacgagaaggagaagaccgccgccggcgccgggaacgtctgcccgcagttcttcaaggtcttcttccccgagctgtcgggggagcggttgaaaattccacccatgttccgccagcacctccaggagcagcctactggaccagtttctctgagaggtcctagtggcaagaaatggcaggcaacacttgcttcagagtccgaagcgtggttctttgagcagggatggaaggaattcgtgacggatcactccatcaacaaaggttacttcttggttttcacatatgatgggccctctcagttctccgtggtggtgttctctccgtcaggcgtcaccgacccaatagctttgaaggcaaagcctacaaatgaggtagtcgttaagattgaagaggatgaagaaggcatccaaggggacatggatgcaggtggcgcctctgaggtaccaattccccccacacaagagggcaatgtagtaacaggaaggagaacaagagcaatgactgctggcgcctctgagataccacctctccccacagaagagggcaatgttgtgacaggaaaaagaacaagagcagtgactgatctcccagcagatgccgatgcatcaaagaggattcaagctgacaagaggaggtctcaagctggaacttcaaaggatgggctgacaattgttcgcaatgccaccttcagtttgttggacgaatctaagactttcaacaagactcaggtcagagataagaatgtgcctaggagtggaaaattccttttgaagaccagcagggcaccggttgtaatatctcaaaggcgcccggtaactgaagaggagagggaccttgctcttagaaaggcgaatgaatttaagtcaaagtaccctttcaccgtgcaaataatgatggaatcttatgtctatgtgggatttttcatgaacatcgcttgcgattttgtcagaaagtcacttccccacacaaacaagaagatgacgctctgggatcccctagggaagccttgggaagttaactatgtttactacagtgaccgctctgttggctctttcagtggtggctggggtaaatttgctctaggaaacaatctggagaagtttgatgtctgcatctttgagcttttcaaggaggacaacataaaagtgcacatatacagggttgtcccagagataactcccctcctccgtgccagcaacaaagactag

SEQ ID NO: 148 - protein, 594 aa, Sorghum bicolor
  misc_feature: Ceres ANNOT ID no.6053768
MAPLRISSSEDGKWERRPTPARTSGLLSRLSKFRIRRRPSKFHGFILHNGKILTTKGIRQLVFIASERNSAAIFIASLSVKSKNTLKNTLTTWRGIQPALLPDGDYLISEKRTTMRGRPKKQQGTTSSAAAAVKEREAARGRPRSKGKETEMGGEGQEPATEKQKKTQGKGMEKEKEKEKNTAVREDGEQDDNNEKEKTAAGAGNVCPQFFKVFFPELSGERLKIPPMFRQHLQEQPTGPVSLRGPSGKKWQATLASESEAWFFEQGWKEFVTDHSINKGYFLVFTYDGPSQFSVVVFSPSGVTDPIALKAKPTNEVVVKIEEDEEGIQGDMDAGGASEVPIPPTQEGNVVTGRRTRAMTAGASEIPPLPTEEGNVVTGKRTRAVTDLPADADASKRIQADKRRSQAGTSKDGLTIVRNATFSLLDESKTFNKTQVRDKNVPRSGKFLLKTSRAPVVISQRRPVTEEERDLALRKANEFKSKYPFTVQIMMESYVYVGFFMNIACDFVRKSLPHTNKKMTLWDPLGKPWEVNYVYYSDRSVGSFSGGWGKFALGNNLEKFDVCIFELFKEDNIKVHIYRVVPEITPLLRASNKD

SEQ ID NO: 149 - DNA, 915 bp, Sorghum bicolor
  misc_feature: Ceres ANNOT ID no.6074550
atggttgaaaaggggtgtgagaggtgcaaggagtggcaggagcactactactgggagcacatggatgtcagcaagatccgcttcttcaagcgcatgacaggagattttgctcatcgcactagcataccggagaagtttgtgaagaaattcaatgggcagatcaccgaaggtgttgacatgaaaacacccagtggtgaaacatggcacgttggtgtagccaagaatcaggatgaactattcttcgtgtctggatgggaggactttgtcaaggcacatgaactgaaggagaatgacctccttctcttcacatgcagaggcagctcttcctttcaggtggtcatctttgaagggagtggctgtgagaaagtgtcttctgtttttggaaacagatttgggaaacaacctgaatctccaaacagtaatagtgactatgatgtcaagcaacaaggaacccgtgaagaggatagtgatagtgacaacgaatacactgacttcaagtactactactcgaggactgccaatcgactgagcaatgacgaaaaaagtaaaataatcagtttggcttcgatccaacctgacaatcctgcgtttattactgtcctacagatgaccaatgtccgccctaggaacaactctctgaccatccccagacaatttgctgctgatcatcttgaggagagatcacaagagatcatacttcgtaggccaaacaggaaagacaagtggtatgtcaggtactattacacgacagccatccgaagccttaaaagctaccactggtccaagtttgtgcgcgaaaacaagcttcgcgagggtgacatctgcgtcttcgaactgatgaaaggcgcaaagagggttactatgagtgttcacgtcatcaagaaggttgaaaaccggtttgttctgttaggataa

(19) United States
(12) Patent Application Publication
(10) Pub. No.: US 2010/0205688 A1
(43) Pub. Date: Aug. 12, 2010

(54) INCREASING TOLERANCE OF PLANTS TO LOW LIGHT CONDITIONS

(76) Inventors: Shing Kwok, Woodland Hills, CA (US); Ryan Miller, Sacramento, CA (US)

Correspondence Address: FISH & RICHARDSON P.C., P.O. BOX 1022, MINNEAPOLIS, MN, 55440-1022, US

(21) Appl. No.: 12/513,086

(22) PCT Filed: Nov. 2, 2007
(86) PCT No.: PCT/US07/83495

§ 371 Date: Apr. 23, 2010

Related U.S. Application Data
(60) Provisional application No. 60/856,613, filed on Nov. 3, 2006.

(51) Int. Cl. A01H 1/00; C12N 5/10; A01H 5/00

(52) U.S. Cl. 800/278; 435/419; 800/295; 800/298; 800/320

(57) ABSTRACT

Methods and materials for modulating (e.g., increasing or decreasing) low light tolerance in plants are disclosed. For example, nucleic acids encoding polypeptides that confer plants with tolerance to low light are disclosed as well as methods for using such nucleic acids to transform plant cells. Also disclosed are plants having increased low light tolerance and plant products produced from plants having increased low light tolerance.

### CROSS-REFERENCE TO RELATED APPLICATIONS

This application claims priority under 35 U.S.C. 119(e) to U.S. Application Ser. No. 60/856,613, filed on Nov. 3, 2006, the entire contents of which are hereby incorporated by reference.

### TECHNICAL FIELD

This document relates to methods and materials involved in tolerance of plants to low light conditions. For example, this document provides transgenic plants and seeds comprising nucleic acids encoding polypeptides that confer tolerance to conditions of low light irradiance.

### BACKGROUND

Light is the source of energy that fuels plant growth through photosynthesis. Light is also a developmental signal that modulates morphogenesis, such as de-etiolation and the transition to reproductive development. Since plants cannot choose their surroundings, they are forced to adapt their growth to ambient light conditions and have evolved complex mechanisms for monitoring the quantity and quality of the surrounding light. For example, many kinds of plants respond to growth under dense canopies or at high densities by growing faster and taller (Cerdan and Chory (2003) Nature, 423:881). Densely planted crops tend to place energy into stem and petiole elongation to lift the leaves into the sunlight rather than putting energy into storage or reproductive structures. The response to low light conditions negatively affects crop yields by reducing the amount of harvestable products such as seeds, fruits and tubers. In addition, tall spindly plants tend to be less wind resistant and lodge more easily, further reducing crop yield.

There is a continuing need for plants that can thrive under less than optimal environmental conditions. One strategy to improve a plant's ability to withstand suboptimal environmental conditions relies upon traditional plant breeding methods. Another approach involves genetic manipulation of plant characteristics through the introduction of exogenous nucleic acids conferring a desirable trait.

### SUMMARY

This document provides methods and materials related to low light tolerance in plants, plant cells, and seeds. For example, this document provides transgenic plants having increased tolerance to conditions of low light irradiance, nucleic acids used to generate transgenic plants having increased tolerance to low light conditions, and methods for making transgenic plants having increased tolerance to low light conditions. Transgenic plants having increased tolerance to low light conditions can exhibit a reduction in one or more responses typically elicited by low light stress. For example, a low light-tolerant transgenic plant have a hypocotyl length, when exposed to low light conditions, e.g., an irradiance of 0.01 to 20 μmol/m²/s of light, that is shorter than the typical hypocotyl length of corresponding wild-type plants grown under similar conditions. Increasing the tolerance of plants to low light conditions can produce healthier plants and a higher crop yield under conditions of low light irradiance, such as those occurring during high-density cultivation of plants.

In one aspect, a method of modulating the low light tolerance of a plant is provided. The method comprises introducing into a plant cell an isolated nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, where a plant produced from the plant cell exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea.

In another aspect, a method of modulating the low light tolerance of a plant is provided. The method comprises introducing into a plant cell an isolated nucleic acid comprising a regulatory region operably linked to a nucleotide sequence encoding a polypeptide, wherein the HMM bit score of the amino acid sequence of said polypeptide is greater than about 5, said HMM based on the amino acid sequences depicted in one of FIGS. 1-3, where a plant produced from the plant cell exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea.

In one aspect, a method of modulating the low light tolerance of a plant is provided. The method comprises introducing into a plant cell an isolated nucleic acid comprising a regulatory region operably linked to a nucleotide sequence selected from the group consisting of SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, and SEQ ID NO:149, or a fragment thereof, where a plant produced from the plant cell exhibits a phenotypic difference relative to a corresponding control plant under low light conditions. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea.

In another aspect, a method of producing a plant is provided. The method comprises growing a plant cell comprising an exogenous nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, where a plant produced from the plant cell exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be dicotyledonous. The plant can be a member of the genus Brassica, Glycine, Gossypium, Helianthus, Lactuca, or Medicago. The plant can be monocotyledonous. The plant can be a member of the genus Cocos, Elaeis, Oryza, Panicum, Sorghum, or Zea.

In another aspect, a method of producing a plant is provided. The method comprises growing a plant cell comprising an exogenous nucleic acid comprising a regulatory region operably linked to a nucleotide sequence encoding a polypeptide, wherein the HMM bit score of the amino acid sequence of said polypeptide is greater than about 5, said HMM based on the amino acid sequences depicted in one of FIGS. 1-3, where a plant produced from the plant cell exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be dicotyledonous. The plant can be a member of the genus Brassica, Glycine, Gossypium, Helianthus, Lactuca, or Medicago. The plant can be monocotyledonous. The plant can be a member of the genus Cocos, Elaeis, Oryza, Panicum, Sorghum, or Zea.

In another aspect, a method of producing a plant is provided. The method comprises growing a plant cell comprising an exogenous nucleic acid comprising a regulatory region operably linked to a nucleotide sequence selected from the group consisting of SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, and SEQ ID NO:149, or a fragment thereof, where a plant produced from the plant cell exhibits a phenotypic difference relative to a corresponding control plant under low light conditions. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be dicotyledonous. The plant can be a member of the genus Brassica, Glycine, Gossypium, Helianthus, Lactuca, or Medicago. The plant can be monocotyledonous. The plant can be a member of the genus Cocos, Elaeis, Oryza, Panicum, Sorghum, or Zea.

In another aspect, a plant is provided. The plant comprises an exogenous nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, where the plant exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea. Progeny of the plant are also provided, where the progeny exhibit a phenotypic difference relative to a corresponding control plant under low light conditions. Seed, vegetative tissue, and fruit from the plant are also provided. In addition, food products and feed products comprising seed, vegetative tissue, and/or fruit from the plant are provided.

In another aspect, a plant is provided. The plant comprises an exogenous nucleic acid comprising a regulatory region operably linked to a nucleotide sequence encoding a polypeptide, wherein the HMM bit score of the amino acid sequence of said polypeptide is greater than about 5, said HMM based on the amino acid sequences depicted in one of FIGS. 1-3, where the plant exhibits a phenotypic difference relative to a corresponding control plant under low light conditions. The nucleotide sequence can encode a polypeptide comprising an amino acid sequence corresponding to SEQ ID NO:79. The nucleotide sequence can encode a polypeptide comprising an amino acid sequence corresponding to SEQ ID NO:83. The nucleotide sequence can encode a polypeptide comprising an amino acid sequence corresponding to SEQ ID NO:86. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea. Progeny of the plant are also provided, where the progeny exhibit a phenotypic difference relative to a corresponding control plant under low light conditions. Seed, vegetative tissue, and fruit from the plant are also provided. In addition, food products and feed products comprising seed, vegetative tissue, and/or fruit from the plant are provided.

In another aspect, a plant is provided. The plant comprises an exogenous nucleic acid comprising a regulatory region operably linked to a nucleotide sequence selected from the group consisting of SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, or a fragment thereof, where the plant exhibits a phenotypic difference relative to a corresponding control plant under low light conditions. The low light conditions can comprise an irradiance of about 0.01 to about 20 μmol/m²/s of light. The phenotypic difference can comprise a decreased hypocotyl length. The regulatory region can be a promoter. The promoter can be a tissue-preferential, broadly expressing, or inducible promoter. The plant can be a dicot. The plant can be a member of the genus Brassica, Glycine, Gossypium, Helianthus, Lactuca, or Medicago. The plant can be a monocot. The plant can be a member of the genus Cocos, Elaeis, Oryza, Panicum, Sorghum, or Zea. Progeny of the plant are also provided, where the progeny exhibit a phenotypic difference relative to a corresponding control plant under low light conditions. Seed, vegetative tissue, and fruit from the plant are also provided. In addition, food products and feed products comprising seed, vegetative tissue, and/or fruit from the plant are provided.

In another aspect, an isolated nucleic acid molecule is provided. The isolated nucleic acid molecule comprises a nucleotide sequence having 95% or greater sequence identity to a nucleotide sequence selected from the group consisting of SEQ ID NO:88, SEQ ID NO:90, SEQ ID NO:105, SEQ ID NO:107, SEQ ID NO:118, and SEQ ID NO:126.

In another aspect, an isolated nucleic acid is provided. The isolated nucleic acid comprises a nucleotide sequence encoding a polypeptide having 80% or greater sequence identity to an amino acid sequence selected from the group consisting of SEQ ID NO:89, SEQ ID NO:91, SEQ ID NO:106, SEQ ID NO:108, and SEQ ID NO:119.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of Ceres CLONE ID no. 110454 (ME04517; SEQ ID NO:79) with homologous and/or orthologous amino acid sequences gi|54291240 (SEQ ID NO:80) and Ceres CLONE ID no. 18857 (SEQ ID NO:104). FIG. 1 and the other alignment figures provided herein were generated using the program MUSCLE version 3.52

FIG. 2 is an alignment of Ceres ANNOT ID no. At1g16640 (ME17025; SEQ ID NO:83) with homologous and/or orthologous amino acid sequence CeresClone:1090611 (SEQ ID NO:84).

FIG. 3 is an alignment of Ceres ANNOT ID no. At1g32690 (ME16639; SEQ ID NO:86) with homologous and/or orthologous amino acid sequences CeresClone:706980 (SEQ ID NO:87), 1485507 (SEQ ID NO:89), CeresClone:1822715 (SEQ ID NO:91), and gi|50911687 (SEQ ID NO:92).

### DETAILED DESCRIPTION

The invention features methods and materials related to modulating the tolerance of plants to conditions of low light irradiation. The methods can include transforming a plant with a nucleic acid encoding a polypeptide, the expression of which results in increased tolerance to low light conditions. Plants produced using such methods can be grown to produce seeds that, in turn, can be used to grow plants having an increased tolerance to conditions of low light irradiance.

Low light conditions can include conditions under which a plant is irradiated with about 0.01 to 20 μmol/m²/s of white light. Plants grown under low light conditions typically exhibit one or more phenotypic changes, or responses, such as an increase in extension growth. Low light tolerance refers to the ability of a plant to grow under low light irradiance levels while exhibiting a low light response that is less than the corresponding low light response exhibited by a control plant. For example, a plant that is tolerant to low light conditions can exhibit less hypocotyl elongation when exposed to low light conditions than a corresponding control plant grown under similar conditions.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include low light-tolerance polypeptides that, when expressed in a plant, can modulate the tolerance of the plant to conditions of low light irradiation. Modulation of the level of low light tolerance can be either an increase or a decrease in the level of low light tolerance relative to the corresponding level in a control plant. Such polypeptides typically contain at least one domain indicative of low light-tolerance polypeptides, as described in more detail herein. Low light-tolerance polypeptides typically have an HMM bit score that is greater than 5, as described in more detail herein. In some embodiments, low light-tolerance polypeptides have greater than 40% identity to SEQ ID NOs:79, 83, and 86, as described in more detail herein.

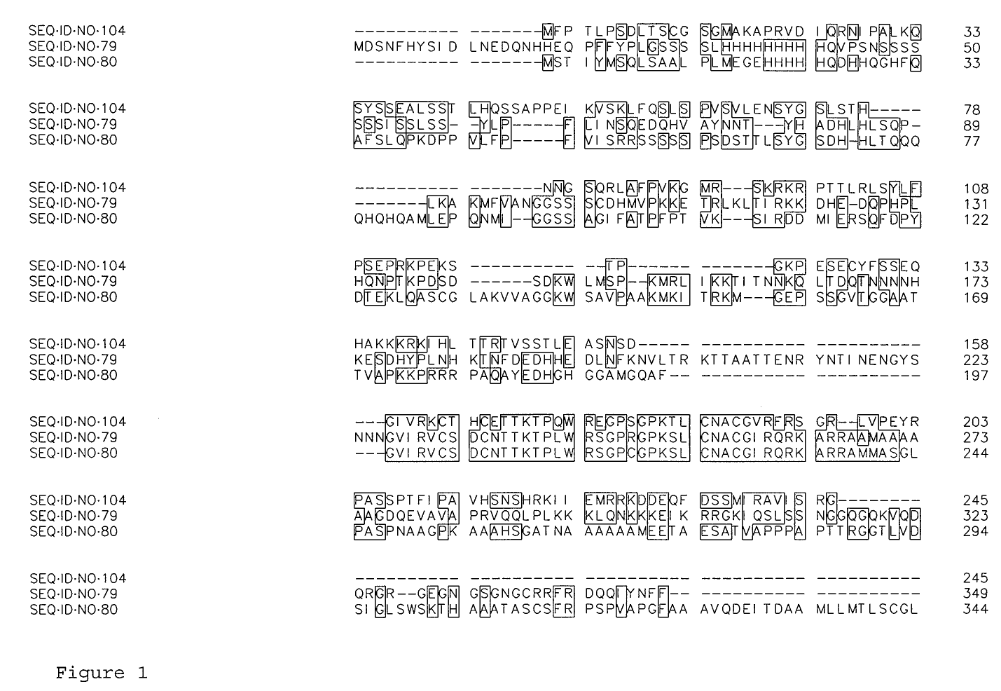

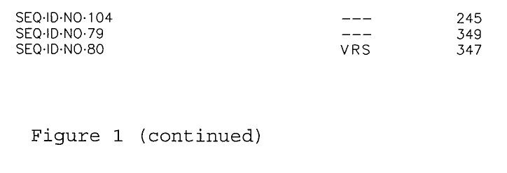

In some embodiments, low light-tolerance polypeptide has an amino acid sequence with at least 40% sequence identity, e.g., 50%, 52%, 56%, 59%, 61%, 65%, 70%, 75%, 80%, 85%, 90%, 95%, 97%, 98%, or 99% sequence identity, to one of the amino acid sequences set forth in SEQ ID NOs:79, 83, and 86. Polypeptides having such a percent sequence identity often have a domain indicative of a low light-tolerance polypeptide and/or have an HMM bit score that is greater than 5, as discussed herein. Amino acid sequences of low light-tolerance polypeptides having at least 50% sequence identity to one of the amino acid sequences set forth in SEQ ID NOs:79, 83, and 86 are provided in FIGS. 1-3.

“Percent sequence identity” refers to the degree of sequence identity between any given reference sequence, e.g., SEQ ID NO:79, and a candidate low light-tolerance sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

A low light-tolerance polypeptide can contain a GATA zinc finger domain. A number of transcription factor polypeptides, including nitrogen regulatory polypeptides, specifically bind the DNA sequence (A/T)GATA(A/G) in the regulatory regions of genes. They are consequently termed GATA-binding transcription factors. The interactions occur via highly-conserved zinc finger domains in which the zinc ion is coordinated by four cysteine residues. Two GATA zinc fingers are found in the GATA transcription factors. There are, however, several proteins which only contain a single copy of the GATA domain. SEQ ID NO:79 sets forth the amino acid sequence of an Arabidopsis clone, identified herein as Ceres CLONE ID no. 110454 (SEQ ID NO:78), that is predicted to encode a polypeptide containing a GATA zinc finger domain.

A low light-tolerance polypeptide can comprise the amino acid sequence set forth in SEQ ID NO:79. Alternatively, a low light-tolerance polypeptide can be a homolog, ortholog, or variant of the polypeptide having the amino acid sequence set forth in SEQ ID NO:79. For example, a low light-tolerance polypeptide can have an amino acid sequence with greater than 70% sequence identity, e.g., 71%, 75%, 80%, 87%, 90%, 95%, 97%, 98%, or 99% sequence identity, to the amino acid sequence set forth in SEQ ID NO:79.

Amino acid sequences of homologs and/or orthologs of the polypeptide having the amino acid sequence set forth in SEQ ID NO:79 are provided in FIG. 1. For example, the alignment in FIG. 1 provides the amino acid sequences of Ceres CLONE ID no. 110454 (SEQ ID NO:79), gi|54291240 (SEQ ID NO:80), and Ceres CLONE ID no. 18857 (SEQ ID NO:104). Other homologs and/or orthologs of SEQ ID NO:79 include gi|15241967 (SEQ ID NO:101), gi|15236131 (SEQ ID NO:102), Ceres CLONE ID no. 1790292 (SEQ ID NO:106), Ceres CLONE ID no. 2007914 (SEQ ID NO:108), gi|125538648 (SEQ ID NO:109), gi|125555801 (SEQ ID NO:110), gi|115445073 (SEQ ID NO:111), gi|115468636 (SEQ ID NO:112), gi|125597642 (SEQ ID NO:113), gi|125581335 (SEQ ID NO:114), Ceres CLONE ID no. 912192 (SEQ ID NO:116), gi|17473547 (SEQ ID NO:117), Ceres CLONE ID no. 1847255 (SEQ ID NO:119), Ceres ANNOT ID no. 6111325 (SEQ ID NO:121), and Ceres ANNOT ID no. 6065280 (SEQ ID NO:123). In some cases, a low light-tolerance polypeptide includes a polypeptide having at least 80% sequence identity, e.g., 80%, 85%, 90%, 95%, 97%, 98%, or 99% sequence identity, to an amino acid sequence corresponding to SEQ ID NO:80, SEQ ID NO: 101, SEQ ID NO: 102, SEQ ID NO: 104, SEQ ID NO: 106, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 116, SEQ ID NO: 117, SEQ ID NO: 119, SEQ ID NO: 121, or SEQ ID NO: 123.

A low light-tolerance polypeptide can contain a B3 DNA binding domain characteristic of a family of plant transcription factor polypeptides with various roles in development. A B3 DNA binding domain is found in VP1/ABI3 transcription factor polypeptides. Some polypeptides, such as RAV1, also have an AP2 DNA binding domain. SEQ ID NO:83 sets forth the amino acid sequence of an Arabidopsis clone, identified herein as Ceres LOCUS ID no. At1g16640 (SEQ ID NO:81), that is predicted to encode a polypeptide containing a B3 DNA binding domain.

A low light-tolerance polypeptide can comprise the amino acid sequence set forth in SEQ ID NO:83. Alternatively, a low light-tolerance polypeptide can be a homolog, ortholog, or variant of the polypeptide having the amino acid sequence set forth in SEQ ID NO:83. For example, a low light-tolerance polypeptide can have an amino acid sequence with greater than 60% sequence identity, e.g., 61%, 62%, 63%, 64%, 67%, 70%, 75%, 80%, 85%, 90%, 95%, 96%, 97%, 98%, or 99% sequence identity, to the amino acid sequence set forth in SEQ ID NO:83.

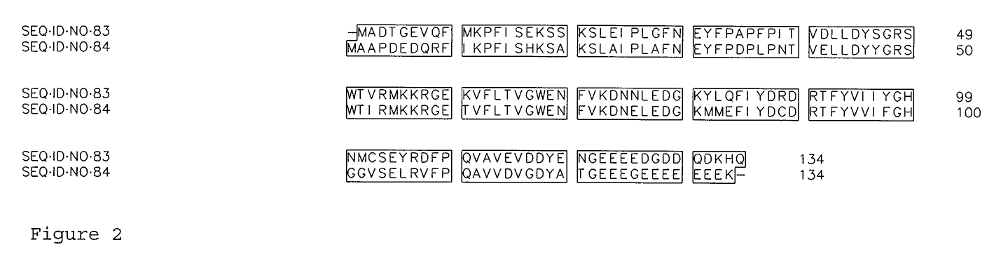

Amino acid sequences of homologs and/or orthologs of the polypeptide having the amino acid sequence set forth in SEQ ID NO:83 are provided in FIG. 2. For example, the alignment in FIG. 2 provides the amino acid sequences of Ceres ANNOT ID no. At1g16640 (SEQ ID NO:83) and CeresClone:1090611 (SEQ ID NO:84). Other homologs and/or orthologs of SEQ ID NO:83 include Ceres ANNOT ID no. 6074550 (SEQ ID NO:124), gi|102139987 (SEQ ID NO:140), gi|6729482 (SEQ ID NO:141), gi|18409814 (SEQ ID NO:142), Ceres CLONE ID no. 36830 (SEQ ID NO:144), Ceres CLONE ID no. 1078253 (SEQ ID NO:146), and Ceres ANNOT ID no. 6053768 (SEQ ID NO:148). In some cases, a low light-tolerance polypeptide includes a polypeptide having at least 80% sequence identity, e.g., 80%, 85%, 90%, 95%, 97%, 98%, or 99% sequence identity, to an amino acid sequence corresponding to SEQ ID NO:84, SEQ ID NO:124, SEQ ID NO:140, SEQ ID NO:141, SEQ ID NO:142, SEQ ID NO:144, SEQ ID NO:146, or SEQ ID NO:148.

SEQ ID NO:86 sets forth the amino acid sequence of an Arabidopsis clone, identified herein as Ceres LOCUS ID no. At1g32690 (SEQ ID NO:85), that is predicted to encode a polypeptide that does not have homology to an existing protein family based on Pfam analysis. A low light-tolerance polypeptide can comprise the amino acid sequence set forth in SEQ ID NO:86. Alternatively, a low light-tolerance polypeptide can be a homolog, ortholog, or variant of the polypeptide having the amino acid sequence set forth in SEQ ID NO:86. For example, a low light-tolerance polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:86.

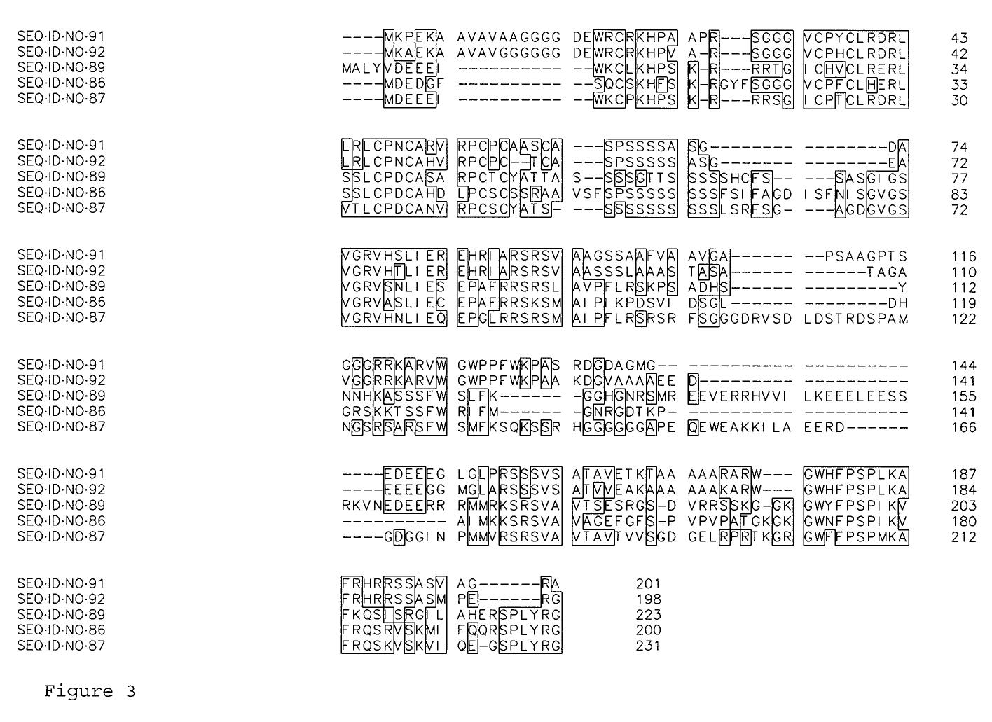

Amino acid sequences of homologs and/or orthologs of the polypeptide having the amino acid sequence set forth in SEQ ID NO:86 are provided in FIG. 3. For example, the alignment in FIG. 3 provides the amino acid sequences of Ceres ANNOT ID no. At1g32690 (SEQ ID NO:86), CeresClone:706980 (SEQ ID NO:87), 1485507 (SEQ ID NO:89), CeresClone:1822715 (SEQ ID NO:91), and gi|50911687 (SEQ ID NO:92). Other homologs and/or orthologs of SEQ ID NO:86 include gi|15226927 (SEQ ID NO:127), gi|28207138 (SEQ ID NO:128), gi|125540748 (SEQ ID NO:129), gi|125549540 (SEQ ID NO:130), gi|115448039 (SEQ ID NO:131), gi|58532121 (SEQ ID NO:132), gi|115460224 (SEQ ID NO:133), gi|125583319 (SEQ ID NO:134), Ceres ANNOT ID no. 6086011, and Ceres ANNOT ID no. 6007163. In some cases, a low light-tolerance polypeptide includes a polypeptide having at least 80% sequence identity, e.g., 80%, 85%, 90%, 95%, 97%, 98%, or 99% sequence identity, to an amino acid sequence corresponding to SEQ ID NO:87, SEQ ID NO:89, SEQ ID NO:91, SEQ ID NO:92, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, or SEQ ID NO:138.

A low light-tolerance polypeptide encoded by a recombinant nucleic acid can be a native low light-tolerance polypeptide, i.e., one or more additional copies of the coding sequence for a low light-tolerance polypeptide that is naturally present in the cell. Alternatively, a low light-tolerance polypeptide can be heterologous to the cell, e.g., a transgenic Lycopersicon plant can contain the coding sequence for a low light-tolerance polypeptide from a Glycine plant.

A low light-tolerance polypeptide can include additional amino acids that are not involved in modulation of low light tolerance, and thus can be longer than would otherwise be the case. For example, a low light-tolerance polypeptide can include an amino acid sequence that functions as a reporter. Such a low light-tolerance polypeptide can be a fusion protein in which a green fluorescent protein (GFP) polypeptide is fused to, e.g., SEQ ID NO:79, or in which a yellow fluorescent protein (YFP) polypeptide is fused to, e.g., SEQ ID NO:83. In some embodiments, a low light-tolerance polypeptide includes a purification tag, a chloroplast transit peptide, a mitochondrial transit peptide, an amyloplast peptide, or a leader sequence added to the amino or carboxy terminus.

Low light-tolerance polypeptide candidates suitable for use in the invention can be identified by analysis of nucleotide and polypeptide sequence alignments. For example, performing a query on a database of nucleotide or polypeptide sequences can identify homologs and/or orthologs of low light-tolerance polypeptides. Sequence analysis can involve BLAST, Reciprocal BLAST, or PSI-BLAST analysis of nonredundant databases using known low light-tolerance polypeptide amino acid sequences. Those polypeptides in the database that have greater than 40% sequence identity can be identified as candidates for further evaluation for suitability as a low light-tolerance polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in low light-tolerance polypeptides, e.g., conserved functional domains.

The identification of conserved regions in a template or subject polypeptide can facilitate production of variants of wild type low light-tolerance polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Amino acid residues corresponding to Pfam domains included in low light-tolerance polypeptides provided herein are set forth in the Sequence Listing. For example, amino acid residues 232 to 267 of the amino acid sequence set forth in SEQ ID NO:79 correspond to a GATA zinc finger domain, as indicated in fields <222> and <223> for SEQ ID NO:79 in the Sequence Listing.

Variants of low light-tolerance polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. A useful variant polypeptide can be constructed based on one of the alignments set forth in FIG. 1, FIG. 2, or FIG. 3. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within low light-tolerance polypeptides. These conserved regions can be useful in identifying functionally similar (orthologous) low light-tolerance polypeptides.

In some instances, suitable low light-tolerance polypeptides can be synthesized on the basis of consensus functional domains and/or conserved regions in polypeptides that are homologous low light-tolerance polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Conserved regions can be identified by homologous polypeptide sequence analysis as described herein. The suitability of polypeptides for use as low light-tolerance polypeptides can be evaluated by functional complementation studies.

### Functional Homologs Identified by HMMER

In some embodiments, low light-tolerance polypeptides include those that fit a Hidden Markov Model based on the polypeptides set forth in any one of FIGS. 1-3. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate low light-tolerance polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The low light-tolerance polypeptides discussed below fit the indicated HMM with an HMM bit score greater than 5 (e.g., greater than 6, 7, 10, 15, 20, 30, 40, 50, 60, 70, 80, 90, 100, 200, 300, 400, or 500). In some embodiments, the HMM bit score of a low light-tolerance polypeptide discussed below is about 50%, 60%, 70%, 80%, 90%, or 95% of the HMM bit score of a functional homolog provided in the Sequence Listing. In some embodiments, a low light-tolerance polypeptide discussed below fits the indicated HMM with an HMM bit score greater than 20, and has a domain indicative of an low light-tolerance polypeptide. In some embodiments, a low light-tolerance polypeptide discussed below fits the indicated HMM with an HMM bit score greater than 20, and has 50% or greater sequence identity (e.g., 75%, 80%, 85%, 90%, 95%, or 100% sequence identity) to an amino acid sequence shown in any one of FIGS. 1-3.

Polypeptides are shown in the Sequence Listing that have HMM bit scores greater than 35 when fitted to an HMM generated from the amino acid sequences set forth in FIG. 1. Such polypeptides include Ceres CLONE ID No. 110454 (SEQ ID NO:79), gi|54291240 (SEQ ID NO:80), gi|15241967 (SEQ ID NO:101), gi|15236131 (SEQ ID NO:102), Ceres CLONE ID no. 18857 (SEQ ID NO:104), Ceres CLONE ID no. 1790292 (SEQ ID NO:106), Ceres CLONE ID no. 2007914 (SEQ ID NO:108), gi|125538648 (SEQ ID NO:109), gi|125555801 (SEQ ID NO:110), gi|115445073 (SEQ ID NO:111), gi|115468636 (SEQ ID NO:112), gi|125597642 (SEQ ID NO:113), gi|125581335 (SEQ ID NO:114), Ceres CLONE ID no. 912192 (SEQ ID NO:116), gi|17473547 (SEQ ID NO:117), Ceres CLONE ID no. 1847255 (SEQ ID NO:119), Ceres ANNOT ID no. 6111325 (SEQ ID NO:121), and Ceres ANNOT ID No. 6065280 (SEQ ID NO:123).

Polypeptides are shown in the Sequence Listing that have HMM bit scores greater than 5 when fitted to an HMM generated from the amino acid sequences set forth in FIG. 2. Such polypeptides include Ceres ANNOT ID No. At1g16640 (SEQ ID NO:83), Ceres Clone ID no. 1090611 (SEQ ID NO:84), Ceres ANNOT ID No. 6074550 (SEQ ID NO:124), gi|1202139987 (SEQ ID NO:140), gi|6729482 (SEQ ID NO:141), gi|18409814 (SEQ ID NO:142), Ceres CLONE ID no. 36830 (SEQ ID NO:144), Ceres CLONE ID no. 1078253 (SEQ ID NO:146), and Ceres ANNOT ID No. 6053768 (SEQ ID NO:148).

Polypeptides are shown in the Sequence Listing that have HMM bit scores greater than 100 when fitted to an HMM generated from the amino acid sequences set forth in FIG. 3. Such polypeptides include Ceres ANNOT ID No. At1g32690 (SEQ ID NO:86), Ceres CLONE ID No. 706980 (SEQ ID NO:87), Ceres ANNOT ID no. 1485507 (SEQ ID NO:89), Ceres CLONE ID no. 1822715 (SEQ ID NO:91), gi|50911687 (SEQ ID NO:92), gi|15226927 (SEQ ID NO:127), gi|28207138 (SEQ ID NO:128), gi|125540748 (SEQ ID NO:129), gi|125549540 (SEQ ID NO:130), gi|115448039 (SEQ ID NO:131), gi|58532121 (SEQ ID NO:132), gi|115460224 (SEQ ID NO:133), gi|125583319 (SEQ ID NO:134), Ceres ANNOT ID no. 6086011 (SEQ ID NO:136), and Ceres ANNOT ID No. 6007163 (SEQ ID NO:138).

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoroamidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to modulate low light tolerance. A recombinant nucleic acid construct comprises a nucleic acid encoding a low light-tolerance polypeptide as described herein, operably linked to a regulatory region suitable for expressing the low light-tolerance polypeptide in the plant or cell. Thus, a nucleic acid can comprise a coding sequence that encodes any of the low light-tolerance polypeptides as set forth in of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148. Examples of nucleic acids encoding low light-tolerance polypeptides are set forth in SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, and SEQ ID NO:149. In some cases, a recombinant nucleic acid construct can include a nucleic acid comprising less than the full-length coding sequence of a low light-tolerance polypeptide. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given low light-tolerance polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Mountain View, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below as well as additional promoters are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; PCT/US05/011105; PCT/US05/034308; and PCT/US05/23639. Nucleotide sequences of promoters are set forth in SEQ ID NOs:1-77 and SEQ ID NOs:93-100. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:75), YP0144 (SEQ ID NO:54), YP0190 (SEQ ID NO:58), p13879 (SEQ ID NO:74), YP0050 (SEQ ID NO:34), p32449 (SEQ ID NO:76), 21876 (SEQ ID NO:1), YP0158 (SEQ ID NO:56), YP0214 (SEQ ID NO:60), YP0380 (SEQ ID NO:69), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:54), YP0380 (SEQ ID NO:69), and PT0585 (SEQ ID NO:4).

Vascular Tissue Promoters

Examples of promoters that have high or preferential activity in vascular bundles include YP0087 (SEQ ID NO:93), YP0093 (SEQ ID NO:94), YP0108 (SEQ ID NO:95), YP0022 (SEQ ID NO:96), and YP0080 (SEQ ID NO:97). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:69), PT0848 (SEQ ID NO:26), YP0381 (SEQ ID NO:70), YP0337 (SEQ ID NO:65), PT0633 (SEQ ID NO:7), YP0374 (SEQ ID NO:67), PT0710 (SEQ ID NO:18), YP0356 (SEQ ID NO:66), YP0385 (SEQ ID NO:72), YP0396 (SEQ ID NO:73), YP0388 (SEQ ID NO:98), YP0384 (SEQ ID NO:71), PT0688 (SEQ ID NO:15), YP0286 (SEQ ID NO:64), YP0377 (SEQ ID NO:68), PD1367 (SEQ ID NO:77), and PD0901 (SEQ ID NO:99). Examples of nitrogen-inducible promoters include PT0863 (SEQ ID NO:27), PT0829 (SEQ ID NO:23), PT0665 (SEQ ID NO:10), and PT0886 (SEQ ID NO:29). Examples of shade-inducible promoters include PR0924 (SEQ ID NO:100), and PT0678 (SEQ ID NO:13).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential such as PT0678 (SEQ ID NO:13), and senescence-preferential promoters. Promoters designated YP0086 (SEQ ID NO:35), YP0188 (SEQ ID NO:57), YP0263 (SEQ ID NO:61), PT0758 (SEQ ID NO:22), PT0743 (SEQ ID NO:21), PT0829 (SEQ ID NO:23), YP0119 (SEQ ID NO:48), and YP0096 (SEQ ID NO:38), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a low light-tolerance polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques As used herein, a transgenic plant also refers to progeny of an initial transgenic plant provided the progeny inherits the transgene. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous low light-tolerance polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

### Plant Species

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, foxglove, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, pear, peas, pecan, pepper, pistachio, plum, poplar, poppy, potato, pumpkin, oilseed rape, quinoa, rapeseed (high erucic acid and canola), safflower, sesame, soaptree bark, soybean, spinach, strawberry, sugar beet, sunflower, sweet potatoes, tea, tomato, walnut, and yams, as well as monocots such as banana, barley, bluegrass, coconut, corn, date palm, fescue, field corn, garlic, millet, oat, oil palm, onion, palm kernel oil, pineapple, popcorn, rice, rye, ryegrass, Sorghum, sudangrass, sugarcane, sweet corn, switchgrass, turf grasses, timothy, and wheat. Gymnosperms such as fir, pine, and spruce can also be suitable.

Thus, the methods and compositions described herein can be used with dicotyledonous plants belonging, for example, to the orders Apiales, Arecales, Aristolochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malpighiales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ephedrales, Ginkgoales, Gnetales, Taxales, and Pinales.

The methods and compositions can be used over a broad range of plant species, including species from the dicot genera Abelmoschus, Acer, Acokanthera, Aconitum, Aesculus, Alangium, Alchornea, Alexa, Alseodaphne, Amaranthus, Ammodendron, Anabasis, Anacardium, Andrographis, Angophora, Anisodus, Apium, Apocynum, Arabidopsis, Arachis, Argemone, Artemisia, Asclepias, Atropa, Azadirachta, Beilschmiedia, Berberis, Bertholletia, Beta, Betula, Bixa, Bleekeria, Borago, Brassica, Calendula, Camellia, Camptotheca, Canarium, Cannabis, Capsicum, Carthamus, Carya, Catharanthus, Centella, Cephaelis, Chelidonium, Chenopodium, Chrysanthemum, Cicer, Cichorium, Cinchona, Cinnamomum, Cissampelos, Citrus, Citrullus, Cocculus, Cocos, Coffea, Cola, Coleus, Convolvulus, Coptis, Corylus, Corymbia, Crambe, Crotalaria, Croton, Cucumis, Cucurbita, Cuphea, Cytisus, Datura, Daucus, Dendromecon, Dianthus, Dichroa, Digitalis, Dioscorea, Duguetia, Erythroxylum, Eschscholzia, Eucalyptus, Euphorbia, Euphoria, Ficus, Fragaria, Galega, Gelsemium, Glaucium, Glycine, Glycyrrhiza, Gossypium, Helianthus, Heliotropium, Hemsleya, Hevea, Hydrastis, Hyoscyamus, Jatropha, Juglans, Lactuca, Landolphia, Lavandula, Lens, Linum, Litsea, Lobelia, Luffa, Lupinus, Lycopersicon, Macadamia, Mahonia, Majorana, Malus, Mangifera, Manihot, Meconopsis, Medicago, Menispermum, Mentha, Micropus, Nicotiana, Ocimum, Olea, Origanum, Papaver, Parthenium, Persea, Petunia, Phaseolus, Physostigma, Pilocarpus, Pistacia, Pisum, Poinsettia, Populus, Prunus, Psychotria, Pyrus, Quillaja, Rabdosia, Raphanus, Rauwolfia, Rhizocarya, Ricinus, Rosa, Rosmarinus, Rubus, Rubia, Salix, Salvia, Sanguinaria, Scopolia, Senecio, Sesamum, Simmondsia, Sinapis, Sinomenium, Solanum, Sophora, Spinacia, Stephania, Strophanthus, Strychnos, Tagetes, Tanacetum, Theobroma, Thymus, Trifolium, Trigonella, Vaccinium, Vicia, Vigna, Vinca, and Vitis; and the monocot genera Agrostis, Allium, Alstroemeria, Ananas, Andropogon, Areca, Arundo, Asparagus, Avena, Cocos, Colchicum, Convallaria, Curcuma, Cynodon, Elaeis, Eragrostis, Erianthus, Festuca, Festulolium, Galanthus, Hemerocallis, Hordeum, Lemna, Lolium, Miscanthus, Musa, Oryza, Panicum, Pennisetum, Phalaris, Phleum, Phoenix, Poa, Ruscus, Saccharum, Secale, Sorghum, Spartina, Triticosecale, Triticum, Uniola, Veratrum, Zea, and Zoysia; and the gymnosperm genera Abies, Cephalotaxus, Cunninghamia, Ephedra, Picea, Pinus, Pseudotsuga, and Taxus.

In some embodiments, a plant can be a species selected from Abelmoschus esculentus (okra), Abies spp. (fir), Acer spp. (maple), Allium cepa (onion), Alstroemeria spp., Ananas comosus (pineapple), Andrographis paniculata, Andropogon gerardii (big bluestem), Artemisia annua, Arundo donax (giant reed), Atropa belladonna, Avena sativa, bamboo, bentgrass (Agrostis spp.), Berberis spp., Beta vulgaris (sugarbeet), Bixa orellana, Brassica juncea, Brassica napus (canola), Brassica rapa, Brassica oleracea (broccoli, cauliflower, brusselsprouts), Calendula officinalis, Camellia sinensis (tea), Camptotheca acuminate, Cannabis sativa, Capsicum annum (hot & sweet pepper), Carthamus tinctorius (safflower), Catharanthus roseus, Cephalotaxus spp., Chrysanthemum parthenium, Cinchona officinalis, Citrullus lanatus (watermelon), Coffea arabica (coffee), Colchicum autumnale, Coleus forskohlii, Cucumis melo (melon), Cucumis sativus (cucumber), Cucurbita maxima (squash), Cucurbita moschata (squash), Cynodon dactylon (bermuda grass), Datura stomonium, Dianthus caryophyllus (carnation), Digitalis lanata, Digitalis purpurea, Dioscorea spp., Elaeis guineensis (palm), Ephedra sinica, Ephedra spp., Erianthus spp., Erythroxylum coca, Eucalyptus spp. (eucalyptus), Festuca arundinacea (tall fescue), Fragaria ananassa (strawberry), Galanthus wornorii, Glycine max (soybean), Gossypium hirsutum (cotton), Gossypium herbaceum, Helianthus annuus (sunflower), Hevea spp. (rubber), Hordeum vulgare, Hyoscyamus spp., Jatropha curcas (jatropha), Lactuca sativa (lettuce), Linum usitatissimum (flax), Lupinus albus (lupin), Lycopersicon esculentum (tomato), Lycopodium serratum (=Huperzia serrata), Lycopodium spp., Manihot esculenta (cassava), Medicago sativa (alfalfa), Mentha piperita (mint), Mentha spicata (mint), Miscanthus spp., Miscanthus giganteus (miscanthus), Musa paradisiaca (banana), Nicotiana tabacum (tobacco), Oryza sativa (rice), Panicum spp., Panicum virgatum (switchgrass), Papaver somniferum (opium poppy), Papaver orientale, Parthenium argentatum (guayule), Pennisetum glaucum (pearl millet), Pennisetum purpureum (elephant grass), Petunia spp. (petunia), Phalaris arundinacea (reed canarygrass), Pinus spp. (pine), Poinsettia pulcherrima (poinsettia), Populus spp., Populus balsamifera (poplar), Populus tremuloides (aspen), Rauwolfia serpentina, Rauwolfia spp., Ricinus communis (castor), Rosa spp. (rose), Saccharum spp. (energycane), Salix spp. (willow), Sanguinaria canadensis, Scopolia spp., Secale cereale (rye), Solanum melongena (eggplant), Solanum tuberosum (potato), Sorghum spp., Sorghum almum, Sorghum bicolor (Sorghum), Sorghum halapense, Sorghum vulgare, Spartina pectinata (prairie cordgrass), Spinacea oleracea (spinach), Tanacetum parthenium, Taxus baccata, Taxus brevifolia, Theobroma cacao (cocoa), Triticale (wheat×rye), Triticum aestivum (wheat), Uniola paniculata (oats), Veratrum californica, Vinca rosea, Vitis vinifera (grape), and Zea mays (corn).

### Transgenic Plant Phenotypes

A transformed cell, callus, tissue, or plant can be identified and isolated by selecting or screening the engineered plant material for particular traits or activities, e.g., expression of a selectable marker gene or modulation of low-light tolerance. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of the transgene. For example, a population of progeny of a single transformation event can be screened for those plants having a desired level of expression of a low light tolerance polypeptide or nucleic acid. As an alternative, a population of plants comprising independent transformation events can be screened for those plants having increased tolerance to conditions of low light irradiation. Selection and/or screening can be carried out over one or more generations, which can be useful to identify those plants that have a desired trait, such as an increased tolerance to conditions of low light irradiation. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

The phenotype of a transgenic plant can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

The phenotype of a transgenic plant and a corresponding control plant that either lacks the transgene or does not express the transgene can be evaluated under particular environmental conditions. For example, the phenotype of a transgenic plant and a corresponding control plant can be evaluated under conditions of low light irradiance. Low light conditions are conditions under which a plant is exposed to an irradiance of about 0.01 μmol/m²/s of light to about 20 μmol/m²/s of light at room temperature and about 70% relative humidity. For example, conditions under which a plant is exposed to 0.01, 1, 5, 10, 15, or 20 μmol/m²/s of light are low light conditions. Sources of lighting and other equipment appropriate for controlling light conditions are known to those in art.

Low light conditions typically have light of a combination of wavelengths, such as white light. White light can be supplied, e.g., by 32 watt fluorescent bulbs (Sylvania, F032/841/ECO, Danvers, Mass.), providing a red:far-red ratio of 13:1. Red wavelengths typically range from a photon irradiance of about 630 to about 700 nm. Far-red wavelengths typically range from a photon irradiance of about 700 to about 750 nm.

In some embodiments, the phenotype of a transgenic plant is assayed under low light conditions in which there is continuous low light during the light period of a light/dark cycle. Continuous low light conditions can be, for example, 16 hours of irradiance with 0.01 to 20 μmol/m²/s of light alternating with 8 hours of darkness. The phenotype of a transgenic plant is assayed once the plant has been exposed to continuous low light conditions during the light period of the light/dark cycle for seven days.

A transgenic plant comprising an exogenous nucleic acid encoding a low light-tolerance polypeptide can exhibit one or more of the following phenotypic differences relative to a corresponding control plant under low light conditions: decreased extension growth, e.g., decreased petiole length, decreased hypocotyl length, decreased internode spacing, and decreased leaf elongation in cereals; increased leaf development, e.g., increased leaf thickness and reduced leaf area growth; decreased apical dominance, e.g., increased branching and tillering; increased chloroplast development, e.g., increased chlorophyll synthesis and a change in the balance of the chlorophyll a:b ratio; alterations in flowering and seed/fruit production, e.g., an increased rate of flowering, an increase in seed set, and increased fruit development; and an increase in storage organ deposition.

Typically, a difference (e.g., an increase) in a morphological feature in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the dimensions of any individual morphological feature is statistically significant at p<0.01, p<0.005, or p<0.001. A statistically significant difference in, for example, a morphological feature in a transgenic plant compared to the corresponding morphological feature a control plant indicates that (1) expression of the recombinant nucleic acid present in the transgenic plant confers the alteration in the morphological feature and/or (2) the recombinant nucleic acid warrants further study as a candidate for altering the morphological feature in a plant.

One suitable phenotype to measure is hypocotyl length. When wild-type seedlings are grown under low light conditions, the hypocotyl length is typically significantly increased relative to the hypocotyl length found in wild-type seedlings grown under conditions of irradiance with about 100 μmol/m²/s of white light.

Seedlings of a transgenic plant and seedlings of a corresponding control plant that either lacks the transgene or does not express the transgene can be grown under low light conditions and, at the appropriate time, hypocotyl lengths from seedlings of each group can be measured. Under low light conditions, a seedling in which the expression of a low light-tolerance polypeptide is increased can have a significantly shorter hypocotyl length than a seedling of a corresponding control plant. The hypocotyl length can be shorter by at least 10 percent, e.g., 10, 15, 20, 25, 30, 35, 40, 45, 50, 55, 60, 65, 70, 75, or more than 75 percent, as compared to the hypocotyl length of a corresponding control plant.

Transgenic plants provided herein have particular uses in agricultural industries. For example, transgenic plants expressing a low light-tolerance polypeptide provided herein can be superior to corresponding control plants in maintaining development and maturation under low light conditions. Such a trait can increase plant survival and seedling establishment at high plant densities, even in crops when plants are near mature growth stages. Transgenic plants expressing a low light-tolerance polypeptide can be more densely planted than those that are not tolerant to low light conditions. Expression of a low light-tolerant polypeptide in plants can provide increased yields compared to plants that are not low light tolerant and that are grown under similar conditions. In addition, expression of polypeptide conferring low light-tolerance in a plant, such as corn, can ensure reasonable seed set in the event that low light conditions prevail during critical stages of plant development, e.g., pollination.

The materials and methods described herein are useful for modifying biomass characteristics, such as characteristics of biomass renewable energy source plants. A biomass renewable energy source plant is a plant having or producing material (either raw or processed) that comprises stored solar energy that can be converted to fuel. In general terms, such plants comprise dedicated energy crops as well as agricultural and woody plants. Examples of biomass renewable energy source plants include: switchgrass, elephant grass, giant chinese silver grass, energycane, giant reed (also known as wild cane), tall fescue, bermuda grass, Sorghum, napier grass (also known as uganda grass), triticale, rye, winter wheat, shrub poplar, shrub willow, big bluestem, reed canary grass, and corn.

Information that the polypeptides disclosed herein can modulate low light tolerance can be useful in breeding of plants. Based on the effect of disclosed polypeptides on low light tolerance, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in low light tolerance. Those polymorphisms that are correlated with an alteration in low light tolerance can be incorporated into a marker assisted breeding program to facilitate the development of lines that have a desired alteration in low light tolerance. Typically, a polymorphism identified in such a manner is used with polymorphisms at other loci that are also correlated with a desired alteration in low light tolerance.

### Articles of Manufacture

Seeds of transgenic plants described herein can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package.

Plants, plant tissues, and/or seeds from plants grown from seeds having an exogenous nucleic acid encoding a low light-tolerance polypeptide can be used for making products including, without limitation, human and animal foods, textiles, oils, and/or ethanol.

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following DNA clones were isolated from Arabidopsis thaliana plants. Ceres CLONE ID no. 110454 (genomic locus At5g56860; SEQ ID NO:78) is a cDNA clone that is predicted to encode a 349 amino acid polypeptide (SEQ ID NO:79). Ceres LOCUS ID no. At1g16640 (SEQ ID NO:81) is a genomic DNA clone that is predicted to encode a 134 amino acid polypeptide (SEQ ID NO:83). Ceres LOCUS ID no. At1g32690 (SEQ ID NO:85) is a genomic DNA clone that is predicted to encode a 200 amino acid polypeptide (SEQ ID NO:86).

Each of Ceres CLONE ID no. 110454, Ceres LOCUS ID no. At1g16640, and Ceres LOCUS ID no. At1g32690 was cloned into a Ti plasmid vector, CRS 338, containing a phosphinothricin acetyltransferase gene, which confers Finale® resistance to transformed plants. Ceres CLONE ID no. 110454, Ceres LOCUS ID no. At1g16640, and Ceres LOCUS ID no. At1g32690 were each operably linked to a CaMV 35S promoter in the constructs made using the CRS 338 vector. Wild-type Arabidopsis thaliana ecotype Wassilewskija (Ws) plants were transformed separately with each construct. The transformation was performed essentially as described in Bechtold and Pelletier, Methods Mol Biol., 82:259-66 (1998).

Transgenic Arabidopsis lines containing Ceres CLONE ID no. 110454, Ceres LOCUS ID no. At1g16640, or Ceres LOCUS ID no. At1g32690 were designated ME04517, ME17025, or ME16639, respectively. The presence of each vector containing Ceres CLONE ID no. 110454, Ceres LOCUS ID no. At1g16640, or Ceres LOCUS ID no. At1g32690 in the respective transgenic Arabidopsis line transformed with the vector was confirmed by Finale® resistance, polymerase chain reaction (PCR) amplification from green leaf tissue extract, and/or sequencing of PCR products.

The segregation of Finale® resistance for T₂ plants from events -02 and -03 of ME04517 was 3:1 (resistant:sensitive). The segregation of Finale® resistance for T₂ plants from events -03 and -05 of ME17025 was 1:1 (resistant:sensitive) and 3:1 (resistant:sensitive), respectively. The segregation of Finale® resistance for T₂ plants from events -02 and -03 of ME16639 was 3:1 (resistant:sensitive) and 2:1 (resistant:sensitive), respectively.

### Example 2

### Identification of Transgenic Plants Tolerant to Low Light Conditions

Wild-type and transgenic seeds were sterilized, plated on solid 0.5×MS medium containing 5 g/L sucrose, and stratified at 4° C. in the dark for three days. After stratification, plates containing the seeds were allowed to reach room temperature. The plates were then transferred to a Conviron walk-in growth chamber (Controlled Environments Inc., Pambina, N. Dak.) at 22° C. and 70% humidity with a 16:8 hour light:dark cycle. Lighting was supplied by 32 watt fluorescent bulbs (Sylvania, F032/841/ECO, Danvers, Mass.), providing a red:far-red ratio of 13:1. The plates were covered with three layers of shade cloth (New York wire, charcoal fiberglass screen, 857650; Home Depot, Atlanta, Ga.) such that the irradiance was about 10 μmol/m²/s. The plates were rotated daily and monitored for changes in hypocotyl elongation. After 48 hours, the plates were scored for late germinators, which were eliminated from consideration as candidate plants having reduced hypocotyl elongation under low light conditions. Each seedling was transplanted to an 8×8 cm well of a flat containing a total of 18 wells (three wells by six wells) and measuring 24 cm by 48 cm in size.

Seedlings maintained under conditions of irradiance with about 10 μmol/m²/s of light for seven days at 22° C. were analyzed for hypocotyl length. Transgenic seedlings having a shorter hypocotyl length than the hypocotyl length of corresponding wild-type seedlings were selected and analyzed using PCR and DNA sequencing to identify the transgenes. The transgenic Arabidopsis lines ME17025, ME04517, and ME16639, described in Example 1, were identified as having reduced hypocotyl elongation under the low light conditions as compared to wild-type control plants.

### Example 3

### Characterization of the Low Light Tolerance of Seedlings from ME17025 Events

T₂ and T₃ seedlings from events -03 and -05 of ME17025 were grown under low light conditions and evaluated for hypocotyl length as described in Example 2. A Chi-square test was performed to compare transgenic seedlings and corresponding non-transgenic segregants having a short or a long hypocotyl. A hypocotyl having a length similar to the hypocotyl length typically exhibited by wild-type Arabidopsis seedlings grown under normal light conditions (e.g., about 100 μmol/m²/s of white light) was considered a short hypocotyl, whereas a hypocotyl having a length similar to that typically exhibited by wild-type Arabidopsis seedlings grown under low light conditions (e.g., about 10 μmol/m²/s of white light) was considered a long hypocotyl. Wild-type Arabidopsis seeds grown for seven days at 22° C. under conditions of irradiance with about 100 μmol/m²/s of white light and a 16:8 hour light:dark cycle typically form hypocotyls that are about 1-3 mm in length. Under conditions of irradiance with about 10 μmol/m²/s of white light, the hypocotyls typically are about 5-7 mm in length.

Seedlings from events -03 and -05 of ME17025 displayed a short hypocotyl under low light conditions in both the T₂ and T₃ generations, and the transgene was linked to the short hypocotyl phenotype with a confidence level of p<0.05 (Table 1).

TABLE 1
Chi-square comparison of the hypocotyl length of transgenic
seedlings and non-transgenic segregants grown under low light
irradiance (10 μmol/m²/s) for seven days

| | Short | Long | | |
|---|---|---|---|---|
| | Hypo- | Hypo- | Chi- | |
| Plants | cotyl | cotyl | Square | p-value |
| T₂ seedlings from event -03 of | 17 | 0 | 23.96 | 9.83E−07 |
| ME17025 | | | | |
| T₂ non-transgenic segregants of | 4 | 16 | | |
| event -03 of ME17025 | | | | |
| T₃ seedlings from event -03-99 | 57 | 1 | 51.95 | 5.68E−13 |
| of ME17025 | | | | |
| T₃ non-transgenic segregants of | 2 | 11 | | |
| event -03-99 of ME17025 | | | | |
| T₂ seedlings from event -05 of | 30 | 0 | 39.00 | 4.24E−10 |
| ME17025 | | | | |
| T₂ non-transgenic segregants of | 0 | 9 | | |
| event -05 of ME17025 | | | | |
| T₃ seedlings from event -05-99 | 56 | 0 | 54.71 | 1.40E−13 |
| of ME17025 | | | | |
| T₃ non-transgenic segregants of | 2 | 10 | | |
| event -05-99 of ME17025 | | | | |

There were no observable or statistically significant differences between T₂ ME17025 plants grown under normal light conditions and control plants in germination, onset of flowering, rosette area, or fertility. The general morphology/architecture appeared wild-type in all instances.

### Example 4

### Characterization of the Low Light Tolerance of Seedlings from ME04517 Events

T₃ and T₄ seedlings from event -02-99 of ME04517, and T₂ and T₃ seedlings from event -03 of ME04517 were grown under low light conditions and evaluated for hypocotyl length as described in Example 2. A Chi-square test was performed to compare transgenic seedlings and corresponding non-transgenic segregants having a short or a long hypocotyl, as described in Example 3. Seedlings from events -02-99 and -03 of ME04517 displayed a short hypocotyl under low light conditions in both generations, and the transgene was linked to the short hypocotyl phenotype with a confidence level of p<0.05 (Table 2).

TABLE 2
Chi-square comparison of the hypocotyl length of transgenic
seedlings and non-transgenic segregants grown under low light
irradiance (10 μmol/m²/s) for seven days

| | Short | Long | | |
|---|---|---|---|---|
| | Hypo- | Hypo- | Chi- | |
| Plants | cotyl | cotyl | Square | p-value |
| T₃ seedlings from event -02-99 | 45 | 9 | 31.05 | 2.51E−08 |
| of ME04517 | | | | |
| T₃ non-transgenic segregants of | 1 | 14 | | |
| event -02-99 of ME04517 | | | | |
| T₄ seedlings from event -02-99- | 39 | 3 | 47.52 | 5.44E−12 |
| 01 of ME04517 | | | | |
| T₄ non-transgenic segregants of | 2 | 23 | | |
| event -02-99-01 of ME04517 | | | | |
| T₂ seedlings from event -03 of | 42 | 9 | 38.95 | 4.34E−10 |
| ME04517 | | | | |
| T₂ non-transgenic segregants of | 1 | 5 | | |
| event-03 of ME04517 | | | | |
| T₃ seedlings from event -03-99 | 49 | 6 | 30.47 | 3.40E−08 |
| of ME04517 | | | | |
| T₃ non-transgenic segregants of | 2 | 11 | | |
| event -03-99 of ME04517 | | | | |

There were no observable or statistically significant differences between T₂ ME04517 plants grown under normal light conditions and control plants in germination, onset of flowering, rosette area, or fertility. The general morphology/architecture appeared wild-type in all instances. Although plants from event -03 initially displayed a slight delay in onset of flowering, this phenotype was not observed during subsequent retesting.

### Example 5

### Characterization of the Low Light Tolerance of Seedlings from ME16639 Events

T₂ and T₃ seedlings from events -02 and -03 of ME16639 were grown under low light conditions and evaluated for hypocotyl length as described in Example 2. A Chi-square test was performed to compare transgenic seedlings and corresponding non-transgenic segregants having a short or a long hypocotyl, as described in Example 3. Seedlings from events -02 and -03 of ME16639 displayed a short hypocotyl under low light conditions in both generations, and the transgene was linked to the short hypocotyl phenotype with a confidence level of p<0.05 (Table 3).

TABLE 3
Chi-square comparison of the hypocotyl length of transgenic
seedlings and non-transgenic segregants grown under low light
irradiance (10 μmol/m²/s) for seven days

| | Short | Long | | |
|---|---|---|---|---|
| | Hypo- | Hypo- | Chi- | |
| Plants | cotyl | cotyl | Square | p-value |
| T₂ seedlings from event -02 of | 26 | 5 | 21.57 | 3.42E−06 |
| ME16639 | | | | |
| T₂ non-transgenic segregants of | 0 | 9 | | |
| event -02 of ME16639 | | | | |
| T₃ seedlings from event -02-99 | 53 | 7 | 20.56 | 5.79E−06 |
| of ME16639 | | | | |
| T₃ non-transgenic segregants of | 0 | 4 | | |
| event -02-99 of ME16639 | | | | |
| T₂ seedlings from event -03 of | 21 | 3 | 19.86 | 8.32E−06 |
| ME16639 | | | | |
| T₂ non-transgenic segregants of | 1 | 10 | | |
| event -03 of ME16639 | | | | |
| T₃ seedlings from event -03-99 | 60 | 6 | 40.91 | 1.59E−10 |
| of ME16639 | | | | |
| T₃ non-transgenic segregants of | 0 | 9 | | |
| event -03-99 of ME16639 | | | | |

There were no observable or statistically significant differences between T₂ ME16639 plants grown under normal light conditions and control plants in germination, onset of flowering, rosette area, or fertility. The general morphology/architecture appeared wild-type in all instances.

### Example 6

### Determination of Functional Homolog and/or Ortholog Sequences

A candidate sequence was considered a functional homolog of a reference sequence if the candidate and reference sequences encoded proteins having a similar function and/or activity. A process known as Reciprocal BLAST (Rivera et al., Proc. Natl. Acad. Sci. USA, 95:6239-6244 (1998)) was used to identify potential functional homolog sequences from databases consisting of all available public and proprietary peptide sequences, including NR from NCBI and peptide translations from Ceres clones.

Before starting a Reciprocal BLAST process, a specific reference polypeptide was searched against all peptides from its source species using BLAST in order to identify polypeptides having BLAST sequence identity of 80% or greater to the reference polypeptide and an alignment length of 85% or greater along the shorter sequence in the alignment. The reference polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog sequence with a specific reference polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a reference polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original reference polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog.

Functional homologs and/or orthologs were identified by manual inspection of potential functional homolog and/or ortholog sequences. Representative functional homologs and/or orthologs for SEQ ID NO:79, SEQ ID NO:83, and SEQ ID NO:86 are shown in FIGS. 1-3, respectively.

### Example 7

### Determination of Functional Homologs by Hidden Markov Models

Hidden Markov Models (HMMs) were generated by the program HMMER 2.3.2. To generate each HMM, the default HMMER 2.3.2 program parameters, configured for glocal alignments, were used.

An HMM was generated using the sequences shown in FIG. 1 as input. These sequences were fitted to the model and a representative HMM bit score for each sequence is shown in the Sequence Listing. Additional sequences were fitted to the model, and representative HMM bit scores for any such additional sequences are shown in the Sequence Listing. The results indicate that these additional sequences are functional homologs of SEQ ID NO:79.

The procedure above was repeated and an HMM was generated for each group of sequences shown in FIGS. 2 and 3, using the sequences shown in each Figure as input for that HMM. A representative bit score for each sequence is shown in the Sequence Listing. Additional sequences were fitted to certain HMMs, and representative HMM bit scores for such additional sequences are shown in the Sequence Listing. The results indicate that these additional sequences are functional homologs of the sequences used to generate that HMM.

### Example 8

### Characterization of the Low Light Tolerance of Seedlings from Orthologous Sequence Events

Ceres CLONE ID no. 18857 (SEQ ID NO:103) was isolated from Arabidopsis thaliana and is predicted to encode a 245 amino acid polypeptide (SEQ ID NO:104). Ceres CLONE ID no. 1090611 (SEQ ID NO:139) was isolated from Brassica napus and is predicted to encode a 134 amino acid polypeptide (SEQ ID NO:84). Ceres CLONE ID no. 706980 (SEQ ID NO:125) was isolated from Glycine max and is predicted to encode a 235 amino acid polypeptide (SEQ ID NO:87).

Each of Ceres CLONE ID no. 18857, Ceres CLONE ID no. 1090611, and Ceres CLONE ID no. 706980 was cloned into a Ti plasmid vector, CRS 338, containing a phosphinothricin acetyltransferase gene, which confers Finale® resistance to transformed plants. Ceres CLONE ID no. 18857, Ceres CLONE ID no. 1090611, and Ceres CLONE ID no. 706980 were each operably linked to a CaMV 35S promoter in the constructs made using the CRS 338 vector. Wild-type Arabidopsis thaliana ecotype Wassilewskija (Ws) plants were transformed separately with each construct. The transformation was performed essentially as described in Bechtold and Pelletier, Methods Mol Biol., 82:259-66 (1998).

Transgenic Arabidopsis lines containing Ceres CLONE ID no. 18857, Ceres CLONE ID no. 1090611, or Ceres CLONE ID no. 706980 were designated ME08493, ME28106, or ME29407, respectively. The presence of each vector containing Ceres CLONE ID no. 18857, Ceres CLONE ID no. 1090611, and Ceres CLONE ID no. 706980 in the respective transgenic Arabidopsis line transformed with the vector was confirmed by Finale® resistance, polymerase chain reaction (PCR) amplification from green leaf tissue extract, and/or sequencing of PCR products.

T₂ seedlings from event -01 ME08493, event -02 of ME28106, and events -01 an -06 of ME29407 were grown under low light conditions and evaluated for hypocotyl length as described in Example 2. A Chi-square test was performed to compare transgenic seedlings and corresponding non-transgenic segregants having a short or a long hypocotyl, as described in Example 3. Seedlings from event -01 ME08493, event -02 of ME28106, and events -01 an -06 of ME29407 displayed a short hypocotyl under low light conditions in both generations, and the transgene was linked to the short hypocotyl phenotype with a confidence level of p<0.05 (Table 4).

TABLE 4
Chi-square comparison of the hypocotyl length of transgenic
seedlings and non-transgenic segregants grown under low light
irradiance (10 μmol/m²/s) for seven days

| | Homolog/ | Short | Long | | |
|---|---|---|---|---|---|
| | ortholog | Hypo- | Hypo- | Chi- | |
| Event | of | cotyl | cotyl | Square | p-value |
| T₂ seedlings from event -01 | ME04517 | 16 | 14 | 5.02 | 2.51E−02 |
| of ME08493 | (SEQ ID | | | | |
| | NO: 79) | | | | |
| T₂ non-transgenic segregants | | 1 | 8 | | |
| of event -01 of ME08493 | | | | | |
| T₂ seedlings from event -02 | ME17025 | 15 | 6 | 7.14 | 7.53E−03 |
| of ME28106 | (SEQ ID | | | | |
| | NO: 83) | | | | |
| T₂ non-transgenic segregants | | 0 | 4 | | |
| of event -02 of ME28106 | | | | | |
| T₂ seedlings from event -01 | ME16639 | 16 | 12 | 5.56 | 1.84E−02 |
| of ME29407 | (SEQ ID | | | | |
| | NO: 86) | | | | |
| T₂ non-transgenic segregants | | 2 | 10 | | |
| of event -01 of ME29407 | | | | | |
| T₂ seedlings from event -06 | | 16 | 11 | 6.82 | 8.99E−03 |
| of ME29407 | | | | | |
| T₂ non-transgenic segregants | | 2 | 11 | | |
| of event -06 of ME29407 | | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modulating the low light tolerance of a plant, said method comprising introducing into a plant cell an exogenous nucleic acid comprising a regulatory region operably lined to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, wherein a plant produced from said plant cell exhibits a phenotypic difference relative to a corresponding control plant under low light conditions.

2. (canceled)

3. A method of modulating the low light tolerance of a plant, said method comprising introducing into a plant cell an exogenous nucleic acid, said exogenous nucleic acid comprising a regulatory region operably linked to a nucleotide sequence having 80 percent or greater sequence identity to a nucleotide sequence selected from the group consisting of SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, and SEQ ID NO:149, or a fragment thereof, wherein a plant produced from said cell exhibits a phenotypic difference relative to a corresponding control plant under low light conditions.

4-6. (canceled)

7. The method of claim 1, wherein said low light conditions comprise irradiance of about 0.01 to about 20 μmol/m²/s of light.

8. The method of claim 1, wherein said a phenotypic difference comprises a decreased hypocotyl length.

9-12. (canceled)

13. The method of claim 1, wherein said plant is a monocot.

14. The method of claim 13, wherein said plant is a member of the genus Cocos, Elaeis, Oryza, Panicum, Sorghum, or Zea.

15. A method of producing a plant, said method comprising growing a plant cell comprising an exogenous nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, wherein a plant produced from said cell exhibits a phenotypic difference relative to a corresponding control plant under low light conditions.

16-20. (canceled)

21. The method of claim 15, wherein said low light conditions comprise irradiance of about 0.01 to about 20 μmol/m²/s of light.

22. The method of claim 15, wherein said a phenotypic difference comprises a decreased hypocotyl length.

23-26. (canceled)

27. The method of claim 15, wherein said plant is a monocot.

28. The method of claim 27, wherein said plant is a member of the genus Cocos, Elaeis, Oryza, Panicum, Sorghum, or Zea.

29. A plant comprising an exogenous nucleic acid, said exogenous nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:83, SEQ ID NO:84, SEQ ID NO:86, SEQ ID NO:87, SEQ ID NO:89, SEQ ID NO:91, SEQ ID NO:92, SEQ ID NO:101, SEQ ID NO:102, SEQ ID NO:104, SEQ ID NO:106, SEQ ID NO:108, SEQ ID NO:109, SEQ ID NO:110, SEQ ID NO:111, SEQ ID NO:112, SEQ ID NO:113, SEQ ID NO:114, SEQ ID NO:116, SEQ ID NO:117, SEQ ID NO:119, SEQ ID NO:121, SEQ ID NO:123, SEQ ID NO:124, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:136, SEQ ID NO:138, SEQ ID NO:140, SEQ ID NO:141, SEQ ID NO:142, SEQ ID NO:144, SEQ ID NO:146, and SEQ ID NO:148, wherein said plant exhibits a phenotypic difference relative to a corresponding control plant under low light conditions.

30. (canceled)

31. A plant comprising an exogenous nucleic acid said exogenous nucleic acid comprising a regulatory region operably linked to a nucleotide sequence having 80 percent or greater sequence identity to a nucleotide sequence selected from the group consisting of SEQ ID NO:78, SEQ ID NO:81, SEQ ID NO:82, SEQ ID NO:85, SEQ ID NO:88, SEQ ID NO:90, SEQ ID NO:103, SEQ ID NO:105, SEQ ID NO:107, SEQ ID NO:115, SEQ ID NO:118, SEQ ID NO:120, SEQ ID NO:122, SEQ ID NO:125, SEQ ID NO:126, SEQ ID NO:135, SEQ ID NO:137, SEQ ID NO:139, SEQ ID NO:143, SEQ ID NO:145, SEQ ID NO:147, and SEQ ID NO:149, or a fragment thereof, wherein said plant exhibits a phenotypic difference relative to a corresponding control plant under low light conditions.

32-34. (canceled)

35. The plant of claim 29, wherein said low light conditions comprise irradiance of about 0.01 to about 20 μmol/m²/s of light.

36. The plant of claim 29, wherein said a phenotypic difference comprises a decreased hypocotyl length.

37-40. (canceled)

41. The plant of claim 29, wherein said plant is a monocot.

42. The plant of claim 41, wherein said plant is a member of the genus Cocos, Elaeis, Oryza, Panicum, Sorghum, or Zea.

43. Progeny of the plant of claim 29,
wherein said progeny exhibit said phenotypic difference relative to said corresponding control plant under low light conditions.

44. (canceled)

45. Vegetative tissue from a plant according to claim 29.

46-50. (canceled)
